(19) United States
(12) Patent Application Publication
(10) Pub. No.: US 2026/0260707 A1
(43) Pub. Date: Sep. 3, 2026

(54) ARTIFICIAL INTELLIGENCE-MEDIATED METHODS AND SYSTEMS FOR GENOME EDITING

(71) Applicant: PIONEER HI-BRED INTERNATIONAL, INC., JOHNSTON, IA (US)

(72) Inventor: ELI RODGERS-MELNICK, JOHNSTON, IA (US)

(73) Assignee: PIONEER HI-BRED INTERNATIONAL, INC., JOHNSTON, IA (US)

(21) Appl. No.: 18/877,635

(22) PCT Filed: Jun. 28, 2023
(86) PCT No.: PCT/US2023/069226

§ 371 Date: Dec. 20, 2024

Related U.S. Application Data
(60) Provisional application No. 63/367,334, filed on Jun. 30, 2022.

(51) Int. Cl. G16B 40/20; C12N 9/22; C12N 9/78; C12N 15/11; C12N 15/82; G16B 20/20

(52) U.S. Cl. CPC G16B 40/20; C12N 9/226; C12N 9/78; C12N 15/11; C12N 15/8213; C12Y 305/04001; C12Y 305/04004; G16B 20/20; C12N 2310/20

(57) ABSTRACT

Described herein are artificial intelligence-mediated methods and systems for genome editing in a plant.

### CROSS-REFERENCE TO RELATED APPLICATION

This application claims priority to U.S. Provisional Application No. 63/367,334, filed Jun. 30, 2022, which is incorporated by reference herein in its entirety.

### BACKGROUND OF THE DISCLOSURE

Every genome contains some number of deleterious mutations, or alleles that when optimized would provide greater fitness to the organism, which together comprise the genetic load. Within the field of plant breeding, selection is traditionally used to improve the desired agronomic phenotypes and thereby gradually purge the genetic load of the breeding population. Agronomic phenotypes such as yield generally have complex genetic architectures, lacking any major single-gene candidates for genome editing. While strong, dominant deleterious variants may be quickly eliminated during the breeding process, slightly deleterious mutations or those with incompletely dominant effects may persist in the breeding population for long periods of time. Moreover, large regions of suppressed recombination within many crop genomes effectively halt purging of individual deleterious variants.

Although several gene editing approaches have been developed for site-specific modification of a plant genome, there still remains a need for more efficient and effective methods for selecting nucleobases in a target DNA sequence for modification by a target genome editing system.

### SUMMARY

In a first aspect, the disclosure provides an artificial intelligence model-mediated method for editing a plant genome, the method comprising: providing an artificial intelligence (AI) model with a first dataset, the first dataset comprising a reference nucleotide sequence of at least one plant regulatory element; providing the AI with a second dataset, the second dataset comprising a plurality of variant nucleotide sequences of the reference nucleotide sequence; predicting one or more expression profiles for each variant nucleotide sequence of the plurality of variant nucleotide sequences relative to expression of the reference nucleotide sequence; calculating a first fitness score for each variant nucleotide sequence, wherein the first fitness score reflects the degree to which a predicted expression profile for a variant nucleotide sequence meets a target expression profile, and wherein the first fitness score incorporates one or more constraints that alter the suitability of the variant nucleotide sequence based on a genome editing system; selecting at least one final variant nucleotide sequence from the plurality of variant nucleotide sequences; and editing the plant genome.

In some aspects of an artificial intelligence model-mediated method for editing a plant genome, prior to selecting at least one final variant nucleotide sequence, the method further comprises: selecting a subset of variant nucleotide sequences based on the first fitness score for each variant nucleotide sequence of the plurality of variant nucleotide sequences; providing the AI model with a third dataset, the third dataset comprising the subset of variant nucleotide sequences, wherein each variant nucleotide sequence of the subset of variant nucleotide sequences comprises one or more additional mutations not found in the plurality of variant nucleotide sequences of the second dataset or results from a recombination of two or more of the variant nucleotide sequences from the second dataset; and optionally repeating the following until a variant nucleotide sequence of the subset of variant nucleotide sequences that meets a target fitness score is identified: predicting one or more expression profiles for each variant nucleotide sequence of the plurality of variant nucleotide sequences relative to expression of the reference nucleotide sequence; calculating a first fitness score for each variant nucleotide sequence; selecting a subset of variant nucleotide sequences based on the first fitness score for each variant nucleotide sequence of the plurality of variant nucleotide sequences; and providing the AI model with a third dataset, the third dataset comprising the subset of variant nucleotide sequences, wherein each variant nucleotide sequence of the subset of variant nucleotide sequences comprises one or more additional mutations not found in the plurality of variant nucleotide sequences of the second dataset or results from a recombination of two or more of the variant nucleotide sequences from the second dataset, wherein the variant nucleotide sequence that meets the target fitness score is selected as a final variant nucleotide sequence.

In some aspects of an artificial intelligence model-mediated method for editing a plant genome, the reference nucleotide sequence is a native or a wild-type nucleotide sequence of the at least one plant regulatory element.

In some aspects of an artificial intelligence model-mediated method for editing a plant genome, editing the plant genome comprises editing a target regulatory element nucleotide sequence in a plant cell such that the target regulatory element nucleotide sequence aligns with the final variant nucleotide sequence.

In some aspects of an artificial intelligence model-mediated method for editing a plant genome, the genome editing system comprises a Cas endonuclease and a guide polynucleotide and editing the target regulatory element nucleotide sequence comprises providing the plant cell with the Cas endonuclease and the guide polynucleotide to introduce at least one site-specific modification in the target regulatory element nucleotide sequence resulting in the variant nucleotide sequence. In some aspects, the genome editing system further comprises a donor DNA. In some aspects, editing the target regulatory element nucleotide sequence comprises introducing at least one nucleotide insertion, at least one nucleotide deletion, at least one nucleotide substitution, or a combination thereof to achieve the final variant nucleotide sequence. In some aspects, the Cas endonuclease is a Cas12 endonuclease or a Cas9 endonuclease.

In some aspects of an artificial intelligence model-mediated method for editing a plant genome, the genome editing system comprises a base editing agent and a plurality of guide polynucleotides and editing the target regulatory element nucleotide sequence comprises providing the plant cell with the base editing agent and the plurality of guide polynucleotides to introduce a plurality of nucleobase edits in the target regulatory element nucleotide sequence resulting in the variant nucleotide sequence. In some aspects, the base editing agent comprises a deactivated Cas endonuclease (dCas) complexed to a deaminase. In some aspects, the deactivated Cas endonuclease is a dCas12f endonuclease or a dCas9 endonuclease and the deaminase is a cytosine deaminase or an adenosine deaminase.

In some aspects of an artificial intelligence model-mediated method for editing a plant genome, the one or more constraints impose a penalty value on the fitness score. In some aspects, the one or more constraints are selected from functions penalizing mutation count, a parsimony constraint, failure to remove a protospacer adjacent motif (PAM) site, lack of a properly positioned PAM site, suboptimal GC content of one or more guide polynucleotides, and distance of a site-specific modification from a DNA break in the target regulatory element nucleotide sequence.

In some aspects of an artificial intelligence model-mediated method for editing a plant genome, the AI model is a natural language processing model, a transformer-based neural network, a convolutional neural network, or a combination thereof.

In some aspects of an artificial intelligence model-mediated method for editing a plant genome, the genome editing system comprises a prime editing agent and one or more guide polynucleotides and editing the target regulatory element nucleotide sequence comprises providing the plant cell with the prime editing agent and the one or more guide polynucleotides to introduce at least one site-specific modification in the target regulatory element nucleotide sequence resulting in the variant nucleotide sequence. In some aspects, the prime editing agent comprises a deactivated Cas endonuclease (dCas) complexed to a reverse transcriptase. In some aspects, the prime editing agent comprises a fusion polypeptide comprising a deactivated Cas endonuclease (dCas) and a reverse transcriptase. In some aspects, the deactivated Cas endonuclease is a dCas 12f endonuclease or a dCas9 endonuclease. In some aspects, the guide polynucleotide is guide RNA. In some aspects, the at least one site-specific modification is an insertion, a deletion, or a substitution (base-to-base conversion).

In another aspect, the disclosure provides an artificial intelligence method for predicting expression modifications due to genetic variants, the method comprising: providing an artificial intelligence (AI) model with a first dataset, the first dataset comprising a reference nucleotide sequence of at least one plant regulatory element; providing the AI model with a second dataset, the second dataset comprising a plurality of variant nucleotide sequences of the reference nucleotide sequence; predicting one or more expression profiles of each variant nucleotide sequence of the plurality of variant nucleotide sequences relative to expression of the reference nucleotide sequence; and calculating a first fitness score for each variant nucleotide sequence, wherein the first fitness score reflects the degree to which a predicted expression profile for a variant nucleotide sequence meets a target expression profile, and wherein the first fitness score incorporates one or more constraints that alter the suitability of the variant nucleotide sequence.

In some aspects of an artificial intelligence method for predicting expression modifications due to genetic variants, the method further comprises: selecting a subset of variant nucleotide sequences based on the first fitness score for each variant nucleotide sequence of the plurality of variant nucleotide sequences; providing the AI model with a third dataset, the third dataset comprising the subset of variant nucleotide sequences, wherein each variant nucleotide sequence of the subset of variant nucleotide sequences comprises one or more additional mutations not found in the plurality of variant nucleotide sequences of the second dataset or results from a recombination of two or more of the variant nucleotide sequences from the second dataset; and optionally repeating the following until a variant nucleotide sequence of the subset of variant nucleotide sequences that meets a target fitness score is identified: predicting one or more expression profiles for each variant nucleotide sequence of the plurality of variant nucleotide sequences relative to expression of the reference nucleotide sequence; calculating a first fitness score for each variant nucleotide sequence; selecting a subset of variant nucleotide sequences based on the first fitness score for each variant nucleotide sequence of the plurality of variant nucleotide sequences; and providing the AI model with a third dataset, the third dataset comprising the subset of variant nucleotide sequences, wherein each variant nucleotide sequence of the subset of variant nucleotide sequences comprises one or more additional mutations not found in the plurality of variant nucleotide sequences of the second dataset or results from a recombination of two or more of the variant nucleotide sequences from the second dataset, wherein the variant nucleotide sequence that meets the target fitness score is selected as a final variant nucleotide sequence.

In some aspects of an artificial intelligence method for predicting expression modifications due to genetic variants, the reference nucleotide sequence is a native or a wild-type nucleotide sequence of the plant regulatory element.

In some aspects of an artificial intelligence method for predicting expression modifications due to genetic variants, the one or more constraints impose a penalty value on the fitness score. In some aspects, the method further comprises defining the one or more constraints based on a genome editing system.

In some aspects of an artificial intelligence method for predicting expression modifications due to genetic variants, the genome editing system comprises a Cas endonuclease and a guide polynucleotide; or a base editing agent and a plurality of guide polynucleotides. In some aspects, the base editing agent comprises a deactivated Cas endonuclease (dCas) complexed to a deaminase. In some aspects, the Cas endonuclease is a Cas12f endonuclease or a Cas9 endonuclease. In some aspects, the dCas endonuclease is a dCas12f endonuclease or a dCas9 endonuclease and the deaminase is a cytosine deaminase or an adenosine deaminase.

In some aspects of an artificial intelligence method for predicting expression modifications due to genetic variants, the one or more constraints are selected from the group functions penalizing mutation count, a parsimony constraint, failure to remove a protospacer adjacent motif (PAM) site, lack of a properly positioned PAM site, suboptimal GC content of one or more guide polynucleotides, and distance of a site-specific modification from a DNA break in the target regulatory element nucleotide sequence.

In some aspects of an artificial intelligence method for predicting expression modifications due to genetic variants, the AI model is a natural language processing model, a transformer-based neural network, a convolutional neural network, or a combination thereof.

In yet another aspect, the disclosure provides an artificial intelligence model-mediated method for breeding genetically modified plants, the method comprising: calculating a fitness score for each of a plurality of variant nucleotide sequences of a plant regulatory element, wherein calculating the fitness score comprises providing an artificial intelligence (AI) model with a first dataset comprising a reference nucleotide sequence of the plant regulatory element and a second dataset comprising the plurality of variant nucleotide sequences of the plant regulatory element and predicting one or more expression profiles of each of the plurality of variant nucleotide sequences relative to expression of the reference nucleotide sequence; selecting a variant nucleotide sequence from the plurality of variant nucleotide sequences based on the fitness score; providing a plant cell with a genome editing system that edits a target regulatory element nucleotide sequence of the plant cell such that the target regulatory element nucleotide sequence aligns with the selected variant nucleotide sequence; regenerating a genetically modified first plant from the plant cell; and crossing the genetically modified first plant with a second plant to produce a population of genetically modified plants.

In some aspects of an artificial intelligence model-mediated method for breeding genetically modified plants, prior to selecting the variant nucleotide sequence from the plurality of variant nucleotide sequences, the method further comprises: (a) predicting one or more expression profiles for each variant nucleotide sequence of the plurality of variant nucleotide sequences relative to expression of the reference nucleotide sequence; (b) calculating an initial fitness score for each of the plurality of variant nucleotide sequences, wherein the fitness score reflects the degree to which a predicted expression profile for a variant nucleotide sequence meets a target expression profile, and wherein the fitness score incorporates one or more constraints that alter the suitability of the variant nucleotide sequence based on a genome editing system; (c) selecting a subset of variant nucleotide sequences based on the initial fitness score for each variant nucleotide sequence of the plurality of variant nucleotide sequences; (d) providing the AI model with a third dataset, the third dataset comprising the subset of variant nucleotide sequences, wherein each variant nucleotide sequence of the subset of variant nucleotide sequences comprises one or more additional mutations not found in the plurality of variant nucleotide sequences in the second dataset or results from a recombination of two or more of the variant nucleotide sequences from the second dataset; (e) optionally repeating (a)-(d) until a variant nucleotide sequence of the subset of variant nucleotide sequences that meets a target fitness score is identified; and (f) selecting at least one final variant nucleotide sequence from the subset of variant nucleotide sequences, wherein the at least one final variant nucleotide sequence meets the target fitness score.

In some aspects of an artificial intelligence model-mediated method for breeding genetically modified plants, the reference nucleotide sequence is a native or a wild-type nucleotide sequence of the plant regulatory element.

In some aspects of an artificial intelligence model-mediated method for breeding genetically modified plants, the genome editing system comprises a Cas endonuclease and a guide polynucleotide that introduce at least one site-specific modification in the target regulatory element nucleotide sequence of the plant cell resulting in the selected variant nucleotide sequence. In some aspects, the genome editing system further comprises a donor DNA. In some aspects, the at least one site-specific modification comprises an insertion, a deletion, a substitution, or a combination thereof. In some aspects, the Cas endonuclease is a Cas12f endonuclease or a Cas9 endonuclease.

In some aspects of an artificial intelligence model-mediated method for breeding genetically modified plants, the genome editing system comprises a base editing agent and a plurality of guide polynucleotides that introduce a plurality of nucleobase edits in the target regulatory element nucleotide sequence. In some aspects, the base editing agent comprises a deactivated Cas endonuclease (dCas) complexed to a deaminase. In some aspects, the deactivated Cas endonuclease is a dCas12f endonuclease or a dCas9 endonuclease and the deaminase is a cytosine deaminase or an adenosine deaminase.

In some aspects of an artificial intelligence model-mediated method for breeding genetically modified plants, calculating the fitness score further comprises imposing a penalty value on the fitness score based on one or more constraints. In some aspects, the one or more constraints are selected from the group functions penalizing mutation count, a parsimony constraint, failure to remove a protospacer adjacent motif (PAM) site, lack of a properly positioned PAM site, suboptimal GC content of one or more guide polynucleotides, and distance of a site-specific modification from a DNA break in the target regulatory element nucleotide sequence.

In some aspects of an artificial intelligence model-mediated method for breeding genetically modified plants, the AI model is a natural language processing model, a transformer-based neural network, a convolutional neural network, or a combination thereof.

In some aspects of an artificial intelligence model-mediated method for breeding genetically modified plants, the genome editing system comprises a prime editing agent and one or more guide polynucleotides and editing the target regulatory element nucleotide sequence comprises providing the plant cell with the prime editing agent and the one or more guide polynucleotides to introduce at least one site-specific modification in the target regulatory element nucleotide sequence resulting in the variant nucleotide sequence. In some aspects, the prime editing agent comprises a deactivated Cas endonuclease (dCas) complexed to a reverse transcriptase. In some aspects, the prime editing agent comprises a fusion polypeptide comprising a deactivated Cas endonuclease (dCas) and a reverse transcriptase. In some aspects, the deactivated Cas endonuclease is a dCas12f endonuclease or a dCas9 endonuclease. In some aspects, the guide polynucleotide is guide RNA.

In a further aspect, the disclosure provides a method for editing a plant genome, the method comprising editing the plant genome to introduce a plurality of site-specific nucleobase edits, wherein the plurality of site-specific edits are selected by one or more artificial intelligence models provided with a first dataset comprising a reference nucleotide sequence of at least one plant regulatory element and a second dataset comprising a plurality of variant nucleotide sequences of the reference nucleotide sequence and configured to select a variant nucleotide sequence from the plurality of variant nucleotide sequences based on one or more expression profiles of the plurality of variant nucleotide sequences relative to expression of the reference nucleotide sequence.

In some aspects of a method for editing a plant genome, editing the plant genome comprises editing a target regulatory element nucleotide sequence in a plant cell such that the target regulatory element nucleotide sequence aligns with the selected variant nucleotide sequence.

In some aspects of a method for editing a plant genome, editing the target regulatory element nucleotide sequence comprises multiplex base editing with a base editing agent and a plurality of guide polynucleotides.

In some aspects of a method for editing a plant genome, the method further comprises providing the plant cell with the base editing agent and the plurality of guide polynucleotides to introduce the plurality of site-specific edits in the target regulatory element nucleotide sequence resulting in the selected variant nucleotide sequence.

In some aspects of a method for editing a plant genome, the base editing agent comprises a deactivated Cas endonuclease (dCas) complexed to a deaminase. In some aspects, the deactivated Cas endonuclease is a dCas12f endonuclease or a dCas9 endonuclease and the deaminase is a cytosine deaminase or an adenosine deaminase.

In some aspects of a method for editing a plant genome, the multiplex base editing introduces at least 10 site-specific nucleobase edits, alternatively at least 100 site-specific nucleobase edits, alternatively at least site-specific 1000 nucleobase edits.

In yet a further aspects, the disclosure provides a system for predicting expression of genetic variants, the system comprising a computer-readable medium comprising an artificial intelligence (AI) model, wherein the AI is configured to: calculate a fitness score for each of a plurality of variant nucleotide sequences of a plant regulatory element, wherein calculating the fitness score comprises providing the AI model with a first dataset comprising a reference nucleotide sequence of the plant regulatory element and a second dataset comprising the plurality of variant nucleotide sequences of the plant regulatory element and predicting one or more expression profiles of each of the plurality of variant nucleotide sequences relative to expression of the reference nucleotide sequence; and select a variant nucleotide sequence from the plurality of variant nucleotide sequences based on the fitness score.

In some aspects of a system for predicting expression of genetic variants, prior to selecting the variant nucleotide sequence from the plurality of variant nucleotide sequences, the system is configured to: (a) predict one or more expression profiles for each variant nucleotide sequence of the plurality of variant nucleotide sequences relative to expression of the reference nucleotide sequence; (b) calculate an initial fitness score for each of the plurality of variant nucleotide sequences, wherein the fitness score reflects the degree to which a predicted expression profile for a variant nucleotide sequence meets a target expression profile, and wherein the fitness score incorporates one or more constraints that alter the suitability of the variant nucleotide sequence based on a genome editing system; (c) select a subset of variant nucleotide sequences based on the initial fitness score for each variant nucleotide sequence of the plurality of variant nucleotide sequences; (d) provide the AI model with a third dataset, the third dataset comprising the subset of variant nucleotide sequences, wherein each variant nucleotide sequence of the subset of variant nucleotide sequences comprises one or more additional mutations not found in the plurality of variant nucleotide sequences in the second dataset or results from a recombination of two or more of the variant nucleotide sequences from the second dataset; (e) optionally repeat (a)-(d) until a variant nucleotide sequence of the subset of variant nucleotide sequences that meets a target fitness score is identified; and (f) select at least one final variant nucleotide sequence from the subset of variant nucleotide sequences, wherein the at least one final variant nucleotide sequence meets the target fitness score.

In some aspects of a system for predicting expression of genetic variants, the system further comprises a computing device comprising a processor.

In some aspects of a system for predicting expression of genetic variants, the reference nucleotide sequence is a native or a wild-type nucleotide sequence of the plant regulatory element.

In some aspects of a system for predicting expression of genetic variants, the AI model incorporates one or more constraints to calculate the fitness score. In some aspects, the one or more constraints are based on a genome editing system and impose a penalty value on the fitness score.

In some aspects of a system for predicting expression of genetic variants, the genome editing system comprises a Cas endonuclease, a guide polynucleotide, and optionally a donor DNA. In some aspects, the Cas endonuclease is a Cas12f endonuclease or a Cas9 endonuclease.

In some aspects of a system for predicting expression of genetic variants, the genome editing system comprises a base editing agent and a plurality of guide polynucleotides. In some aspects, the base editing agent comprises a deactivated Cas endonuclease (dCas) complexed to a deaminase. In some aspects, the deactivated Cas endonuclease is a dCas12f endonuclease or a dCas9 endonuclease and the deaminase is a cytosine deaminase or an adenosine deaminase.

In some aspects of a system for predicting expression of genetic variants, the selected variant nucleotide sequence comprises nucleobase edits for multiplex base editing of a plant genome.

In some aspects of a system for predicting expression of genetic variants, the selected variant nucleotide sequence comprises at least 10 nucleobase edits, alternatively at least 100 nucleobase edits, alternatively at least 1000 nucleobase edits.

In some aspects of a system for predicting expression of genetic variants, the one or more constraints are selected from the group functions penalizing mutation count, a parsimony constraint, failure to remove a protospacer adjacent motif (PAM) site, lack of a properly positioned PAM site, suboptimal GC content of one or more guide polynucleotides, and distance of a site-specific modification from a DNA break in the target regulatory element nucleotide sequence.

In some aspects of a system for predicting expression of genetic variants, the AI model is a natural language processing model, a transformer-based neural network, a convolutional neural network, or a combination thereof.

In some aspects of a system for predicting expression of genetic variants, the genome editing system comprises a prime editing agent and one or more guide polynucleotides. In some aspects, the prime editing agent comprises a deactivated Cas endonuclease (dCas) complexed to a reverse transcriptase. In some aspects, the prime editing agent comprises a fusion polypeptide comprising a deactivated Cas endonuclease (dCas) and a reverse transcriptase. In some aspects, the deactivated Cas endonuclease is a dCas12f endonuclease or a dCas9 endonuclease. In some aspects, the guide polynucleotide is guide RNA.

In yet another aspect, the disclosure provides an artificial intelligence model-mediated method for editing a microbial genome, the method comprising: providing an artificial intelligence (AI) model with a first dataset, the first dataset comprising a reference nucleotide sequence of a microbial genome; providing the AI with a second dataset, the second dataset comprising a plurality of variant nucleotide sequences of the reference nucleotide sequence; predicting one or more expression profiles for each variant nucleotide sequence of the plurality of variant nucleotide sequences relative to expression of the reference nucleotide sequence; calculating a first fitness score for each variant nucleotide sequence, wherein the first fitness score reflects the degree to which a predicted expression profile for a variant nucleotide sequence meets a target expression profile, and wherein the first fitness score incorporates one or more constraints that alter the suitability of the variant nucleotide sequence based on a genome editing system; selecting at least one final variant nucleotide sequence from the plurality of variant nucleotide sequences; and editing the microbial genome.

In some aspects of an artificial intelligence model-mediated method for editing a microbial genome, prior to selecting the at least one final variant nucleotide sequence, the method further comprises: selecting a subset of variant nucleotide sequences based on the first fitness score for each variant nucleotide sequence of the plurality of variant nucleotide sequences; providing the AI model with a third dataset, the third dataset comprising the subset of variant nucleotide sequences, wherein each variant nucleotide sequence of the subset of variant nucleotide sequences comprises one or more additional mutations not found in the plurality of variant nucleotide sequences of the second dataset or results from a recombination of two or more of the variant nucleotide sequences from the second dataset; and optionally repeating the following until a variant nucleotide sequence of the subset of variant nucleotide sequences that meets a target fitness score is identified: predicting one or more expression profiles for each variant nucleotide sequence of the plurality of variant nucleotide sequences relative to expression of the reference nucleotide sequence; calculating a first fitness score for each variant nucleotide sequence; selecting a subset of variant nucleotide sequences based on the first fitness score for each variant nucleotide sequence of the plurality of variant nucleotide sequences; and providing the AI model with a third dataset, the third dataset comprising the subset of variant nucleotide sequences, wherein each variant nucleotide sequence of the subset of variant nucleotide sequences comprises one or more additional mutations not found in the plurality of variant nucleotide sequences of the second dataset or results from a recombination of two or more of the variant nucleotide sequences from the second dataset, wherein the variant nucleotide sequence that meets the target fitness score is selected as a final variant nucleotide sequence.

In some aspects of an artificial intelligence model-mediated method for editing a microbial genome, editing the microbial genome comprises editing a target nucleotide sequence in a microbial cell such that the target nucleotide sequence aligns with the final variant nucleotide sequence.

In some aspects of an artificial intelligence model-mediated method for editing a microbial genome, the genome editing system comprises a Cas endonuclease and a guide polynucleotide and editing the target nucleotide sequence comprises providing the cell with the Cas endonuclease and the guide polynucleotide to introduce at least one site-specific modification in the target nucleotide sequence resulting in the variant nucleotide sequence. In some aspects, the genome editing system further comprises a donor DNA. In some aspects, editing the target nucleotide sequence comprises introducing at least one nucleotide insertion, at least one nucleotide deletion, at least one nucleotide substitution, or a combination thereof to achieve the final variant nucleotide sequence. In some aspects, the Cas endonuclease is a Cas12 endonuclease or a Cas9 endonuclease.

In some aspects of an artificial intelligence model-mediated method for editing a microbial genome, the genome editing system comprises a base editing agent and a plurality of guide polynucleotides and editing the target nucleotide sequence comprises providing the cell with the base editing agent and the plurality of guide polynucleotides to introduce a plurality of nucleobase edits in the target nucleotide sequence resulting in the variant nucleotide sequence. In some aspects, the base editing agent comprises a deactivated Cas endonuclease (dCas) complexed to a deaminase. In some aspects, the deactivated Cas endonuclease is a dCas12f endonuclease or a dCas9 endonuclease and the deaminase is a cytosine deaminase or an adenosine deaminase.

In some aspects of an artificial intelligence model-mediated method for editing a microbial genome, the one or more constraints impose a penalty value on the fitness score. In some aspects, the one or more constraints are selected from functions penalizing mutation count, a parsimony constraint, failure to remove a protospacer adjacent motif (PAM) site, lack of a properly positioned PAM site, suboptimal GC content of one or more guide polynucleotides, and distance of a site-specific modification from a DNA break in the target nucleotide sequence.

In some aspects of an artificial intelligence model-mediated method for editing a microbial genome, the AI model is a natural language processing model, a transformer-based neural network, a convolutional neural network, or a combination thereof.

In some aspects of an artificial intelligence model-mediated method for editing a microbial genome, the microbial genome is a bacterial, viral, or fungal genome.

In some aspects of an artificial intelligence model-mediated method for editing a microbial genome, the genome editing system comprises a prime editing agent and one or more guide polynucleotides and editing the target nucleotide sequence comprises providing the cell with the prime editing agent and the one or more guide polynucleotides to introduce at least one site-specific modification in the target nucleotide sequence resulting in the variant nucleotide sequence. In some aspects, the prime editing agent comprises a deactivated Cas endonuclease (dCas) complexed to a reverse transcriptase. In some aspects, the prime editing agent comprises a fusion polypeptide comprising a deactivated Cas endonuclease (dCas) and a reverse transcriptase. In some aspects, the deactivated Cas endonuclease is a dCas12f endonuclease or a dCas9 endonuclease. In some aspects, the guide polynucleotide is guide RNA. In some aspects, the at least one site-specific modification is an insertion, a deletion, or a substitution (base-to-base conversion).

In yet another aspect, the disclosure provides an artificial intelligence model-mediated method for editing a non-human mammalian genome, the method comprising: providing an artificial intelligence (AI) model with a first dataset, the first dataset comprising a reference nucleotide sequence of a non-human mammal; providing the AI with a second dataset, the second dataset comprising a plurality of variant nucleotide sequences of the reference nucleotide sequence; predicting one or more expression profiles for each variant nucleotide sequence of the plurality of variant nucleotide sequences relative to expression of the reference nucleotide sequence; calculating a first fitness score for each variant nucleotide sequence, wherein the first fitness score reflects the degree to which a predicted expression profile for a variant nucleotide sequence meets a target expression profile, and wherein the first fitness score incorporates one or more constraints that alter the suitability of the variant nucleotide sequence based on a genome editing system; selecting at least one final variant nucleotide sequence from the plurality of variant nucleotide sequences; and editing the non-human mammalian genome.

In some aspects of an artificial intelligence model-mediated method for editing a non-human mammalian genome, prior to selecting the at least one final variant nucleotide sequence, the method further comprises: selecting a subset of variant nucleotide sequences based on the first fitness score for each variant nucleotide sequence of the plurality of variant nucleotide sequences; providing the AI model with a third dataset, the third dataset comprising the subset of variant nucleotide sequences, wherein each variant nucleotide sequence of the subset of variant nucleotide sequences comprises one or more additional mutations not found in the plurality of variant nucleotide sequences of the second dataset or results from a recombination of two or more of the variant nucleotide sequences from the second dataset; and optionally repeating the following until a variant nucleotide sequence of the subset of variant nucleotide sequences that meets a target fitness score is identified: predicting one or more expression profiles for each variant nucleotide sequence of the plurality of variant nucleotide sequences relative to expression of the reference nucleotide sequence; calculating a first fitness score for each variant nucleotide sequence; selecting a subset of variant nucleotide sequences based on the first fitness score for each variant nucleotide sequence of the plurality of variant nucleotide sequences; and providing the AI model with a third dataset, the third dataset comprising the subset of variant nucleotide sequences, wherein each variant nucleotide sequence of the subset of variant nucleotide sequences comprises one or more additional mutations not found in the plurality of variant nucleotide sequences of the second dataset or results from a recombination of two or more of the variant nucleotide sequences from the second dataset, wherein the variant nucleotide sequence that meets the target fitness score is selected as a final variant nucleotide sequence.

In some aspects of an artificial intelligence model-mediated method for editing a non-human mammalian genome, editing the non-human mammal genome comprises editing a target nucleotide sequence in a non-human mammalian cell such that the target nucleotide sequence aligns with the final variant nucleotide sequence.

In some aspects of an artificial intelligence model-mediated method for editing a non-human mammalian genome, the genome editing system comprises a Cas endonuclease and a guide polynucleotide and editing the target nucleotide sequence comprises providing the cell with the Cas endonuclease and the guide polynucleotide to introduce at least one site-specific modification in the target nucleotide sequence resulting in the variant nucleotide sequence. In some aspects, the genome editing system further comprises a donor DNA. In some aspects, editing the target nucleotide sequence comprises introducing at least one nucleotide insertion, at least one nucleotide deletion, at least one nucleotide substitution, or a combination thereof to achieve the final variant nucleotide sequence. In some aspects, the Cas endonuclease is a Cas12 endonuclease or a Cas9 endonuclease.

In some aspects of an artificial intelligence model-mediated method for editing a non-human mammalian genome, the genome editing system comprises a base editing agent and a plurality of guide polynucleotides and editing the target nucleotide sequence comprises providing the cell with the base editing agent and the plurality of guide polynucleotides to introduce a plurality of nucleobase edits in the target nucleotide sequence resulting in the variant nucleotide sequence. In some aspects, the base editing agent comprises a deactivated Cas endonuclease (dCas) complexed to a deaminase. In some aspects, the deactivated Cas endonuclease is a dCas12f endonuclease or a dCas9 endonuclease and the deaminase is a cytosine deaminase or an adenosine deaminase.

In some aspects of an artificial intelligence model-mediated method for editing a non-human mammalian genome, the one or more constraints impose a penalty value on the fitness score. In some aspects, the one or more constraints are selected from functions penalizing mutation count, a parsimony constraint, failure to remove a protospacer adjacent motif (PAM) site, lack of a properly positioned PAM site, suboptimal GC content of one or more guide polynucleotides, and distance of a site-specific modification from a DNA break in the target nucleotide sequence.

In some aspects of an artificial intelligence model-mediated method for editing a non-human mammalian genome, the AI model is a natural language processing model, a transformer-based neural network, a convolutional neural network, or a combination thereof.

In some aspects of an artificial intelligence model-mediated method for editing a non-human mammalian genome, the non-human mammalian genome is from cattle, sheep, pigs, goats, horses, mules, cats, dogs, rabbits, rats, or mice.

In some aspects of an artificial intelligence model-mediated method for editing a non-human mammalian genome, the genome editing system comprises a prime editing agent and one or more guide polynucleotides and editing the target nucleotide sequence comprises providing the cell with the prime editing agent and the one or more guide polynucleotides to introduce at least one site-specific modification in the target nucleotide sequence resulting in the variant nucleotide sequence. In some aspects, the prime editing agent comprises a deactivated Cas endonuclease (dCas) complexed to a reverse transcriptase. In some aspects, the prime editing agent comprises a fusion polypeptide comprising a deactivated Cas endonuclease (dCas) and a reverse transcriptase. In some aspects, the deactivated Cas endonuclease is a dCas 12f endonuclease or a dCas9 endonuclease. In some aspects, the guide polynucleotide is guide RNA. In some aspects, the at least one site-specific modification is an insertion, a deletion, or a substitution (base-to-base conversion).

### DESCRIPTION OF THE FIGURES

FIG. 1A is a graph illustrating k-mer predictive accuracy for held-out chromosomes of training genomes in a Masked Language Model.

FIG. 1B is a graph illustrating k-mer predictive accuracy for permuted versions of the held-out chromosomes of training genomes in a Masked Language Model.

FIG. 1C is a graph illustrating k-mer predictive accuracy for held-out testing genomes in a Masked Language Model.

FIG. 1D is a graph illustrating k-mer predictive accuracy for permuted versions of the held-out testing genomes in a Masked Language Model.

FIGS. 2A and 2B illustrate a precision-recall curve (left), a receive-operator characteristic plot (middle), and a predicted vs. observed expression plot (right) for held-out genes in 6 maize tissues for predictive performance of a pre-trained transformer-based model backbone with a fine-tuned expression-predicting head.

FIG. 3A illustrates within-gene Pearson R correlations of predicted vs. observed expression for held-out maize genes as observed or after permutation of predicted profiles among expressed genes.

FIG. 3B illustrates the relationship between tissue-tissue expression correlations in a predicted testing set vs. the observed expression correlations the same testing set.

FIG. 4A illustrates the maximum change and position of maximum effect in predicted expression of testing set genes following insertion of the canonical TATA box or a permuted TATA box sequence.

FIG. 4B illustrates distribution of the maximal changes in predicted expression of a testing set gene following insertion of the canonical TATA box nucleotide sequence or the permuted TATA box nucleotide sequence.

FIG. 4C illustrates distribution of the maximal changes in predicted expression of a testing set gene following insertion of a dual copy of the TCP element nucleotide sequence or a dual copy of the permuted TCP element nucleotide sequence.

FIG. 4D illustrates distribution of the maximal changes in predicted expression of a testing set gene following insertion of a dual copy of the HSF element nucleotide sequence or a dual copy of the permuted HSF element nucleotide sequence.

FIG. 4E illustrates distribution of the maximal changes in predicted expression of a testing set gene following insertion of a CMV35S 90 bp nucleotide sequence or a permuted CMV35S 90 bp nucleotide sequence.

FIG. 5 is a schematic illustrating a genetic algorithm comprising an expression prediction model according to some aspects of the disclosure.

### DETAILED DESCRIPTION

The disclosures herein are described more fully hereinafter with reference to the accompanying figures, in which some, but not all possible aspects are shown. Indeed, disclosures may be embodied in many different forms and should not be construed as limited to the aspects set forth herein; rather, these aspects are provided so that this disclosure will satisfy applicable legal requirements.

Many modifications and other aspects disclosed herein will come to mind to one skilled in the art to which the disclosed methods and compositions pertain having the benefit of the teachings presented in the following descriptions and the associated figures. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” can include the aspect of “consisting of.” Unless defined otherwise, all technical and scientific terms used herein have the same meaning as commonly understood by one of ordinary skill in the art to which the disclosed methods and compositions belong. In this specification and in the claims which follow, reference is made to a number of terms which shall be defined herein.

As used herein the singular forms “a”, “an”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this disclosure belongs unless clearly indicated otherwise.

The present disclosure provides methods and systems for artificial intelligence-mediated genome editing of plants and plants. The methods and systems described herein provide a precise means of modulating or modifying plant gene expression, wherein the modifications encompass constitutive or transient upregulation of gene expression, constitutive or transient downregulation of gene expression, and/or alteration of relative tissue expression levels. More specifically, the methods and systems of the present disclosure modify target polynucleotide sequences (e.g., polynucleotide sequences of plant regulatory elements) by endonuclease-mediated base editing or endonuclease-mediated homologous recombination. Site-specific modifications to target polynucleotide sequences result from predictive expression analytics provided by the artificial intelligence models of the disclosure, which predict and identify suitable variant polynucleotide sequences of target polynucleotide sequences based on a genome editing system. Further, the methods and systems described herein can provide artificial intelligence-mediated genome editing of microbial genomes and non-human mammalian genomes.

Plants that can be used with the methods and systems descried herein include, but are not limited to, monocots such as corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses; dicots such as soybean (Glycine max), Brassica species (for example but not limited to: oilseed rape or Canola) (Brassica napus, B. campestris, Brassica rapa, Brassica juncea), alfalfa (Medicago sativa), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), potato (Solanum tuberosum); and other plants including safflower (Carthamus tinctorius), sweet potato (Ipomoea batatus), cassava (Manihot esculenta), coffee (Coffea spp.), coconut (Cocos nucifera), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), vegetables, ornamentals, and conifers. Vegetables that can be used include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that can be used include pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow cedar (Chamaecyparis nootkatensis).

As used herein, “expression” refers to the production of a functional end-product (e.g., an mRNA, guide polynucleotide, or a protein) in either precursor or mature form.

As used herein, “plant” generally refers to whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. Plant cells comprise a plant cell wall, and as such are distinct, with different biochemical characteristics, from protoplasts that lack a cell wall.

A “plant element” or “plant part” A “plant element” or “plant part” is intended to reference either a whole plant or a plant component, which may comprise differentiated and/or undifferentiated tissues, for example but not limited to plant tissues, parts, and cell types. In some aspects, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue), plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides. The term “plant organ” refers to plant tissue or a group of tissues that constitute a morphologically and functionally distinct part of a plant. As used herein, a “plant element” is synonymous to a “portion” or “part” of a plant, and refers to any part of the plant, and can include distinct tissues and/or organs, and may be used interchangeably with the term “tissue” throughout. Similarly, a “plant reproductive element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keiki, or bud. The plant element may be in plant or in a plant organ, tissue culture, or cell culture.

The term “monocotyledonous” or “monocot” refers to the subclass of angiosperm plants also known as “monocotyledoneae”, whose seeds typically comprise only one embryonic leaf, or cotyledon. The term includes references to whole plants, plant elements, plant organs (e.g., leaves, stems, roots, etc.), seeds, plant cells, and progeny of the same.

The term “dicotyledonous” or “dicot” refers to the subclass of angiosperm plants also knows as “dicotyledoneae”, whose seeds typically comprise two embryonic leaves, or cotyledons. The term includes references to whole plants, plant elements, plant organs (e.g., leaves, stems, roots, etc.), seeds, plant cells, and progeny of the same.

As used herein, “crossed”, “cross”, “crossing” refers to the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

As used herein “target site,” “target sequence,” “target DNA,” “target locus,” “genomic target site,” “target polynucleotide sequence”, and “target nucleotide sequence” are used interchangeably and refer to a polynucleotide sequence in the genome (including choloroplastic and mitochondrial DNA) of a plant cell at which a nick, single-strand break, or double-strand break is induced in a plant cell genome by an endonuclease (e.g., Cas endonuclease). The target site is an endogenous site in the plant genome, or alternatively, the target site is heterologous to the plant and thereby not naturally occurring in the genome, or the target site is found in a heterologous genomic location compared to where it occurs in nature.

As used herein, “endogenous target nucleotide sequence”, “native target nucleotide sequence”, and “wild-type nucleotide sequence” are used interchangeably herein to refer to a target nucleotide sequence that is endogenous or native to the genome of a plant and is at the endogenous or native position of that target sequence in the genome of the plant.

An “artificial target site” or “artificial target sequence” are used interchangeably herein and refer to a target nucleotide sequence that has been introduced into the genome of a plant. Such an artificial target sequence is identical in sequence to an endogenous or native target sequence in the genome of a plant but is located in a different position (i.e., a non-endogenous or non-native position) in the genome of a plant.

An “altered target site,” “altered target sequence” “modified target site,” and “modified target sequence” are used interchangeably herein and refer to a target nucleotide sequence as disclosed herein that comprises at least one alteration when compared to non-altered target sequence. Such “alterations” or “modifications” include, for example: (i) replacement of at least one nucleotide, (ii) a deletion of at least one nucleotide, (iii) an insertion of at least one nucleotide, or (iv) any combination of (i)-(iii).

As used herein “targeted mutation”, “targeted modification”, “site-specific mutation”, and “site-specific modification” are used interchangeably and refer to a mutation in a target polynucleotide sequence, including native polynucleotide sequences, that was made by altering the target polynucleotide sequence using the methods and systems described herein.

In a first aspect, the disclosure provides artificial intelligence-mediated methods for editing a plant genome. In some aspects of an artificial intelligence-mediated method for editing a plant genome, the method includes providing an artificial intelligence (AI) model with a first dataset, the first data set comprising a reference nucleotide sequence of a plant regulatory element or at least one plant regulatory element; providing the artificial intelligence model with a second dataset, the second dataset comprising one or more variant nucleotide sequences of the reference nucleotide sequence; predicting one or more expression profiles of the one or more variant nucleotide sequences relative to expression of the reference nucleotide sequence; calculating a fitness score for each variant nucleotide sequence; selecting at least one variant nucleotide sequence; and editing the plant genome such that the target regulatory element nucleotide sequence in a plant cell or plant aligns with the selected variant nucleotide sequence. In some aspects of the artificial intelligence-mediated method for editing a plant genome, the fitness score reflects the degree to which a predicted expression profile for a variant nucleotide sequence meets a target expression profile. In some aspects of the artificial intelligence-mediated method for editing a plant genome, the fitness score incorporates one or more constraints that alter the suitability of a variant nucleotide sequence. In some aspects of the artificial intelligence-mediated method for editing a plant genome, the one or more constraints that alter the suitability of a variant nucleotide sequence are based on a target or pre-selected genome editing system.

In some aspects of the artificial intelligence-mediated method for editing a plant genome, calculating a fitness score for a variant nucleotide sequence or each variant nucleotide sequence of a plurality of variant nucleotide sequences further comprises selecting a subset of variant nucleotide sequences based on an initial fitness score for each variant nucleotide sequence; providing the AI model with a third dataset, the third dataset comprising the subset of variant nucleotide sequences, wherein each variant nucleotide sequence of the subset of variant nucleotide sequences comprises an additional mutation or mutations not found in the corresponding variant nucleotide sequence of the one or more variant nucleotide sequences of the second dataset; and optionally repeating the steps of predicting one or more expression profiles of the one or more variant nucleotide sequences relative to expression of the reference nucleotide sequence and calculating a fitness score for each variant nucleotide sequence until a variant nucleotide sequence of the subset of variant nucleotide sequences that meets a target fitness score is identified, wherein the variant nucleotide sequence that meets the target fitness score is selected as a final variant nucleotide sequence.

In a second aspect, the disclosure provides artificial intelligence methods for predicting expression modifications due to genetic variants. In some aspects of an artificial intelligence method for predicting expression modifications due to genetic variants, the method includes providing an artificial intelligence model with a first dataset, the first dataset comprising a reference nucleotide sequence of a plant regulatory element or at least one plant regulatory element; providing the artificial intelligence model with a second dataset comprising one or more variant nucleotide sequences of the reference nucleotide sequence; predicting one or more expression profiles of the one or more variant nucleotide sequences relative to expression of the reference nucleotide sequence; and calculating a fitness score for the variant nucleotide sequence or each of the variant nucleotide sequences. In some aspects of the artificial intelligence method for predicting expression modifications due to genetic variants, the fitness score reflects the degree to which a predicted expression profile for a variant nucleotide sequence meets a target expression profile. In some aspects of the artificial intelligence method for predicting expression modifications due to genetic variants, the fitness score incorporates one or more constraints that alter the suitability of a variant nucleotide sequence. In some aspects of the artificial intelligence method for predicting expression modifications due to genetic variants, the one or more constraints that alter the suitability of a variant nucleotide sequence are based on a target or pre-selected genome editing system.

In some aspects of the artificial intelligence method for predicting expression modifications due to genetic variants, calculating a fitness score for a variant nucleotide sequence or each variant nucleotide sequence of a plurality of variant nucleotide sequences further comprises selecting a subset of variant nucleotide sequences based on an initial fitness score for each variant nucleotide sequence; providing the AI model with a third dataset, the third dataset comprising the subset of variant nucleotide sequences, wherein each variant nucleotide sequence of the subset of variant nucleotide sequences comprises an additional mutation or mutations not found in the corresponding variant nucleotide sequence of the plurality of variant nucleotide sequences of the second dataset; and optionally repeating the steps of predicting one or more expression profiles of the one or more variant nucleotide sequences relative to expression of the reference nucleotide sequence and calculating a fitness score for each variant nucleotide sequence until a variant nucleotide sequence of the subset of variant nucleotide sequences that meets a target fitness score is identified, wherein the variant nucleotide sequence that meets the target fitness score is selected as a final variant nucleotide sequence.

In a third aspect, the disclosure provides artificial intelligence-mediated methods for breeding genetically modified plants. In some aspects of an artificial intelligence-mediated method for breeding genetically modified plants, the method includes calculating a fitness score for one or more variant nucleotide sequences of a plant regulatory element or at least one plant regulatory element, wherein calculating the fitness score comprises providing an artificial intelligence model with a first dataset comprising a reference nucleotide sequence of the plant regulatory element and a second dataset comprising one or more variant nucleotide sequences of the plant regulatory element (or plant regulatory elements) and predicting one or more expression profiles of the one or more variant nucleotide sequences relative to expression of the reference nucleotide sequence; selecting at least one variant nucleotide sequence based on the fitness score; providing a plant cell with a genome editing system that edits a target regulatory element nucleotide sequence of the plant cell such that the target regulatory element nucleotide sequence aligns with the selected variant nucleotide sequence; regenerating a genetically modified first plant from the plant cell; and crossing the genetically modified first plant with a second plant to produce a population of genetically modified plants.

In some aspects of the artificial intelligence-mediated method for breeding genetically modified plants, calculating a fitness score for each variant nucleotide sequence comprises (a) predicting one or more expression profiles for each variant nucleotide sequence relative to expression of the reference nucleotide sequence; (b) calculating an initial fitness score for each of the variant nucleotide sequences, wherein the fitness score reflects the degree to which a predicted expression profile for a variant nucleotide sequence meets a target expression profile, and wherein the fitness score incorporates one or more constraints that alter the suitability of the variant nucleotide sequence based on a genome editing system; (c) selecting a subset of variant nucleotide sequences based on the initial fitness score for each variant nucleotide sequence; (d) providing the AI model with a third dataset, the third dataset comprising the subset of variant nucleotide sequences, wherein each variant nucleotide sequence of the subset of variant nucleotide sequences comprises an additional mutation or mutations not found in the corresponding variant nucleotide sequences in the second dataset; (e) optionally repeating (a)-(d) until a variant nucleotide sequence of the subset of variant nucleotide sequences that meets a target fitness score is identified; and (f) selecting at least one final variant nucleotide sequence from the subset of variant nucleotide sequences, wherein the at least one final variant nucleotide sequence meets the target fitness score.

In a fourth aspect, the disclosure provides methods for editing a plant genome that include editing a plant genome to introduce at least one site-specific nucleobase edits or a plurality of site-specific nucleobase edits, wherein the one or more site-specific nucleobase edits are selected by one or more artificial intelligence models provided with a first dataset comprising a reference nucleotide sequence of a plant regulatory element and a second dataset comprising one or more variant nucleotide sequences of the reference nucleotide sequence and configured to select a variant nucleotide sequence from the one or more variant nucleotide sequences based on one or more expression profiles of the one or more variant nucleotide sequences relative to expression of the reference nucleotide sequence.

In a fifth aspect, the disclosure provides systems for predicting expression of genetic variants. In some aspects of a system for predicting expression of genetic variants, the system includes a computer-readable medium comprising an artificial intelligence model or one or more artificial intelligence models, wherein the artificial intelligence model (or the one or more artificial intelligence models) is configured to: calculate a fitness score for one or more variant nucleotide sequences of a plant regulatory element, wherein calculating the fitness score comprises providing the artificial intelligence model with a first dataset comprising a reference nucleotide sequence of the plant regulatory element and a second dataset comprising the one or more variant nucleotide sequences of the plant regulatory element and predicting one or more expression profiles of the one or more variant nucleotide sequences relative to expression of the reference nucleotide sequence; and selecting a variant nucleotide sequence from the one or more variant nucleotide sequences based on the fitness score.

In a sixth aspect, the disclosure provides artificial intelligence-mediated methods for editing a microbial genome. In some aspects of an artificial intelligence-mediated method for editing a microbial genome, the method includes providing an artificial intelligence (AI) model with a first dataset, the first data set comprising a reference nucleotide sequence from a microbial genome, such as a microbial regulatory element; providing the artificial intelligence model with a second dataset, the second dataset comprising one or more variant nucleotide sequences of the reference nucleotide sequence; predicting one or more expression profiles of the one or more variant nucleotide sequences relative to expression of the reference nucleotide sequence; calculating a fitness score for each variant nucleotide sequence; selecting at least one variant nucleotide sequence; and editing the microbial genome such that the target nucleotide sequence in a microbial cell aligns with the selected variant nucleotide sequence.

In some aspects of an artificial intelligence-mediated method for editing a microbial genome, the microbial genome is a bacterial, viral, or fungal genome.

In a seventh aspect, the disclosure provides artificial intelligence-mediated methods for editing a non-human mammalian genome. In some aspects of an artificial intelligence-mediated method for editing a non-human mammalian genome, the method includes providing an artificial intelligence (AI) model with a first dataset, the first data set comprising a reference nucleotide sequence from a non-human mammalian genome, such as a regulatory element; providing the artificial intelligence model with a second dataset, the second dataset comprising one or more variant nucleotide sequences of the reference nucleotide sequence; predicting one or more expression profiles of the one or more variant nucleotide sequences relative to expression of the reference nucleotide sequence; calculating a fitness score for each variant nucleotide sequence; selecting at least one variant nucleotide sequence; and editing the non-human mammalian genome such that the target nucleotide sequence in a non-human mammalian cell aligns with the selected variant nucleotide sequence.

In some aspects of an artificial intelligence-mediated method for editing a non-human mammalian genome, the non-human mammalian genome is from cattle, sheep, pigs, goats, horses, mules, cats, dogs, rabbits, rats, or mice.

In some aspects of the system for predicting expression of genetic variants, calculating a fitness score for each variant nucleotide sequence comprises (a) predicting one or more expression profiles for each variant nucleotide sequence relative to expression of the reference nucleotide sequence; (b) calculating an initial fitness score for each of the variant nucleotide sequences, wherein the fitness score reflects the degree to which a predicted expression profile for a variant nucleotide sequence meets a target expression profile, and wherein the fitness score incorporates one or more constraints that alter the suitability of the variant nucleotide sequence based on a genome editing system; (c) selecting a subset of variant nucleotide sequences based on the initial fitness score for each variant nucleotide sequence; (d) providing the AI model with a third dataset, the third dataset comprising the subset of variant nucleotide sequences, wherein each variant nucleotide sequence of the subset of variant nucleotide sequences comprises an additional mutation or mutations not found in the corresponding variant nucleotide sequences in the second dataset; (e) optionally repeating (a)-(d) until a variant nucleotide sequence of the subset of variant nucleotide sequences that meets a target fitness score is identified; and (f) selecting at least one final variant nucleotide sequence from the subset of variant nucleotide sequences, wherein the at least one final variant nucleotide sequence meets the target fitness score.

As used herein, a “regulatory element”, “plant regulatory element”, “regulatory sequence”, and “regulatory nucleotide sequence” refer to nucleotide sequences located upstream (5′ non-coding sequences), within, or downstream (3′ non-coding sequences) of a coding sequence, and which influence the transcription, RNA processing or stability, and/or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures. Regulatory sequences include known or unknown nucleotide sequences that affect or modulate expression of a coding sequence, for example, the magnitude or spatial-temporal profile of expression. As used herein, “promoter” or “promoter sequence” refers to a region of DNA involved in the recognition and binding of RNA polymerase and other proteins to initiate transcription. A promoter can comprise, but is not required to comprise, a TATA box capable of directing RNA polymerase II to initiate RNA synthesis at the appropriate transcription initiation site for a particular coding sequence. A promoter sequence consists of proximal and more distal upstream elements, the latter elements often referred to as enhancers. As used herein, “enhancer” refers to a DNA sequence that can stimulate promoter activity. Enhancers can be an innate element of the promoter or a heterologous element inserted to enhance the level or tissue-specificity of a promoter. Promoters may be derived in their entirety from a native gene, be composed of different elements derived from different promoters found in nature, and/or comprise synthetic DNA segments. It is understood by those skilled in the art that different promoters can direct the expression of a gene or coding sequence in different tissues or cell types, at different stages of development, or in response to different environmental conditions. It is further recognized that since in most cases the exact boundaries of regulatory sequences have not been completely defined, DNA fragments of some variation may have promoter activity.

As used herein, “heterologous” refers to the difference between the original environment, location, or composition of a particular polynucleotide or polypeptide sequence and its current environment, location, or composition. Non-limiting examples include differences in taxonomic derivation (e.g., a polynucleotide sequence obtained from Zea mays would be heterologous if inserted into the genome of an Oryza sativa plant, or of a different variety or cultivar of Zea mays; or a polynucleotide obtained from a bacterium was introduced into a cell of a plant), or sequence (e.g., a polynucleotide sequence obtained from Zea mays, isolated, modified, and re-introduced into a maize plant). As used herein, “heterologous” in reference to a sequence can refer to a sequence that originates from a different species, variety, foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, one or more regulatory region(s) and/or a polynucleotide provided herein may be entirely synthetic. In one aspect, a discrete component of a poly-gRNA molecule is heterologous to at least one other component, i.e., do not occur together in nature.

As used herein, a “reference sequence” refers to a predetermined sequence used as a basis for sequence comparison. A reference sequence may be a subset or the entirety of a specified sequence; for example, as a segment of a full-length cDNA or gene sequence, or the complete cDNA, gene sequence, or protein sequence. It will be understood that a reference sequence includes protein or polypeptide sequences (i.e., “reference polypeptide sequence” or “reference protein sequence”) and polynucleotide sequences (i.e., “reference polynucleotide sequence” or “reference nucleotide sequence”).

Editing targets of the present disclosure include, but are not limited to, proximal and distal expression control elements for transcriptional, post-transcriptional, and/or translational regulation of gene expression.

For example, editing targets of the methods described herein include promoters, translation leader sequences, 5′ untranslated sequences, 3′ untranslated sequences, introns, polyadenylation target sequences, RNA processing sites, effector binding sites, and stem-loop structures. Editing targets of the present disclosure also include distal expression control elements such as, for example, distal enhancers, distal silencers, insulator elements, 3′-UTR miRNA binding sites, 3′-UTR siRNA binding sites, and 5′-UTR upstream open reading frames (uORFs). The methods described herein can also target sequences for epigenetic regulation such as, for example, long non-coding RNAs (lncRNA), methyltransferases, chromatin remodelers, and histone acetyltransferase/methyltransferase.

In some aspects of the methods and systems disclosed herein (i.e., artificial intelligence-mediated methods for editing a plant genome, artificial intelligence methods for predicting expression modifications in genetic variants, artificial intelligence-mediated methods for breeding genetically modified plants, methods for editing a plant genome that include editing a plant genome to introduce one or more of site-specific edits, and systems for predicting expression of genetic variants), a reference sequence can be a nucleotide sequence of a plant regulatory element. In some aspects, a reference nucleotide sequence is a native or wild-type nucleotide sequence of a plant regulatory element.

Any suitable artificial intelligence model (AI model) can be used in the in the methods and systems described herein. Types of models include, but are not limited to, statistical models, such as probability models, regression models, and those involving deep learning, such as supervised, semi-supervised, and unsupervised models, or combinations thereof. In some aspects, an artificial intelligence model can be a classification model, a regression model, a clustering model, a dimensionality reduction model, retrospective index model, a distribution model, for example, a multivariate or univariate Gaussian distribution model, or a deep learning model. As used herein, “deep learning” refers to a subclass of machine learning and artificial intelligence based on a multi-layered structure of algorithms or artificial neural networks in which multiple layers of processing are used to extract higher level features from data. As used herein “neural network” refers to an actual or simulated (e.g., by computer program) network comprised of numerous, independent, highly interconnected artificial neurons which simulate the functions of biological neurons through a set of algorithms.

In some aspects, the deep learning model can be part of an ensemble model. In some aspects, the deep learning model can be an ensemble model comprising two or more models. In some aspects, the deep learning model can be a supervised learning model, such as a classification or regression model. The artificial intelligence models can include support vector machines, neural networks, such as SVM (Support Vector machines) or ANN (Artificial Neural Networks), or deep learning algorithms and the like.

In some aspects, the artificial intelligence model can incorporate boosting algorithms, random forests or random decision forests, support vector machines, normalizing flows, recurrent neural networks (RNNs), fully dense neural networks, spiking neural networks, and/or generative adversarial networks. As used herein, “support vector machines” describe statistical analyses that determine a boundary (i.e., an n-dimensional hyperplane) which distinguishes between class members using a kernel-associated basis expansion.

In another example, the methods described herein can utilize generative artificial intelligence as implemented through, for example, a transformer-based decoder model, a generative adversarial network (GAN), and/or an autoregressive normalizing flow.

In some aspects of the methods and systems disclosed herein, the artificial intelligence (AI) model is a natural language processing (NLP) model, a transformer-based neural network, a convolutional neural network, or a combination thereof. Neural networks or artificial neural networks (ANN) refer to a set of algorithms comprised of one or more functional compositions of linear or affine transformations and nonlinear-activation functions, used to map between an input and an output space. Natural language processing (NLP) refers to the use of computers to analyze, understand, and derive meaning from human language to organize and structure knowledge for applications such as automatic text summarization, sentiment analysis, topic extraction, named entity recognition, relationship extraction, and stemming. A transformer-based neural network is a deep learning model that differentially weights the significance of each part of input data and tracks relationships in sequential data. Transformer models apply an evolving set of mathematical techniques, called attention or self-attention, to detect subtle ways even distant data elements in a series influence and depend on each other. Like recurrent neural networks (RNNs), transformers process sequential input data, such as natural language, but unlike RNNs, transformers process the entire input at once as the attention mechanism provides context for any position in the input sequence. A convolutional neural network (CNN or ConvNet) is a deep learning model that can take in an input image or sequence and process it through one or more neural network layers, wherein the components of each layer only attend to a locally-contiguous subset of the previous layer. In some aspects, the artificial intelligence model can utilize a hybrid network of transformers to capture long-range dependencies and CNNs to model local features of input data.

In some aspects of the methods and systems disclosed herein, the artificial intelligence model is established or generated from a supervised learning model using one or more data profiles for training or learning (“training data profile”). The one or more training data profiles can be genomic data profiles (or subsets thereof), transcriptomic data profiles, proteomic data profiles, metabolomic data profiles, spectral data profiles, or phenotypic data profiles. The training data profiles can be from a whole plant or from certain plant tissues or parts thereof including seeds, leaves, immature plants or seedlings, such as V4-V10 growth stages. The training data profiles can be obtained from monocot or dicot plants, including but not limited to, soybean, maize, sorghum, cotton, canola, sunflower, rice, wheat, sugarcane, alfalfa tobacco, barley, cassava, peanuts, millet, oil palm, potatoes, rye, or sugar beet plants. Training data profiles can be from inbred, hybrid, or native plants.

A “genomic data profile” generally refers to a set of information about the entire genome of a plant or group of plants, a subset of the genome of a plant or group of plants, or a combination thereof. A genomic data profile can include information regarding the presence or absence in the genome of a specific set of mutations, single nucleotide polymorphisms (SNPs), insertion of nucleobases, deletion of nucleobases, genotypic markers, other sequence information, or any combination thereof.

A “proteomic profile” generally refers to a set of information about all the proteins expressed by a given genome, given cell, given tissue, or a given plant or group of plants at a certain time or it can encompass a specific subset of proteins expressed by a given genome, given cell, given tissue, or a given plant or group of plants at a certain time or any combination thereof. In some aspects, a proteomic profile data includes but is not limited to protein sequences and protein expression data.

A “transcriptomic profile” generally refers to a set of information about all the genes expressed in a given plant or group of plants (genome-wide transcriptomic), or it can encompass a specific subset of genes expressed in a given plant or group of plants or any combination thereof. In some aspects, the level of expression of the genes, temporal expression, spatial expression, or any combination thereof may be included in the transcriptomic profile. In some aspects, the transcriptomic profile data includes but is not limited to RNA transcript sequences and gene expression data by RNA sequence analysis.

As used herein, “fitness score” or “fitness function” generally refers to how close a candidate design solution is to meeting the overall specification of a desired solution. In some aspects of the methods and systems disclosed herein, a fitness score for a variant nucleotide sequence refers to the distance between a variant nucleotide sequence's predicted expression in one or more tissues and/or developmental timepoints and the target expression in those same tissues and/or developmental timepoints as defined by a user or an autonomous agent.

As used herein, a “variant nucleotide sequence” refers to nucleotide sequence derived from a reference nucleotide sequence by deletion or addition of one or more nucleobases at one or more positions in the reference nucleotide sequence and/or substitution of one or more nucleobases at one or more positions in the reference nucleotide sequence. In some aspects of the methods and systems disclosed herein, variant nucleotide sequences can be at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or greater percent sequence identity to the reference nucleotide sequence. “Sequence identity” or “identity” in the context of nucleotide sequences refers to the nucleic acid bases in two sequences that are the same when aligned for maximum correspondence over a specified comparison window.

As used herein, an “expression profile” refers to a mapping of a nucleotide sequence to a set of real numbers associated with the abundance of a product of the nucleotide sequence in a tissue or set of tissues and/or developmental stage under consideration. An expression profile may either be observed through means of a biological assay or predicted by one or more of the artificial intelligence models described herein. The latter case is designated the “predicted expression profile”. An expression profile can further include spatiotemporal expression of a variant nucleotide sequence. In some aspects of the methods and systems disclosed herein, an expression profile refers to the predicted or projected expression magnitude (i.e., transcription) and/or spatiotemporal characteristics of a variant nucleotide sequence, wherein the variant nucleotide sequence is derived from a reference nucleotide sequence of a plant regulatory element.

In some aspects of the methods and systems disclosed herein, the fitness score of a variant nucleotide sequence incorporates one or more constraints or penalties based on a target genome editing system. In this regard the fitness score can be adjusted (i.e., increased or decreased in value) due to a variant nucleotide sequence being more or less matched or suited for a genome editing system. Constraints of the methods and systems disclosed herein include, but are not limited to, functions penalizing mutation count, failure to remove a protospacer adjacent motif (PAM) site, lack of a properly positioned PAM site, suboptimal GC content of one or more guide polynucleotides, and distance of a site-specific modification from a DNA break in the target regulatory element nucleotide sequence.

In some aspects of the methods and systems disclosed herein, “penalizing mutation count” refers to adjusting the fitness score of a variant nucleotide sequence to account for each nucleobase mutation, with each nucleobase mutation resulting in and imposing a penalty on the fitness score. As used herein, a “nucleobase mutation” refers to an insertion, deletion, or substitution of a nucleobase (including C·G to T·A or an A·T to G·C base editing conversions). In some aspects of the methods and systems disclosed herein, a mutation count (i.e., the total number of nucleobase mutations) for a variant nucleotide sequence does not exceed 15 nucleobase changes or mutations.

In some aspects of the methods and systems disclosed herein, a function penalizing mutation count can be a parsimony constraint. As used herein, “parsimony” refers to a variant nucleotide sequence's ability to achieve a target expression profile and/or a predicted expression profile with a minimal number of nucleobase mutations. As used herein, a “parsimony constraint” or “parsimony penalty” refers to a penalty value imposed on the fitness score of a variant nucleotide due to the number of nucleobase mutations (i.e., the mutation count) within the variant nucleotide sequence that are needed to achieve a target expression profile and/or a predicted expression profile. In some aspects, a parsimony constraint applies a penalty to the fitness score of a variant nucleotide sequence if the number of nucleobase mutations in the variant nucleotide sequence exceeds a predetermined threshold. In some aspects, a parsimony constraint applies a penalty to the fitness score of a variant nucleotide sequence for each nucleobase mutation in the variant nucleotide sequence.

In some aspects, the mutation count for a variant nucleotide sequence does not exceed 30 nucleobase changes or mutations, alternatively does not exceed 25 nucleobase changes or mutations, alternatively does not exceed 20 nucleobase changes or mutations, alternatively does not exceed 15 nucleobase changes or mutations, alternatively does not exceed 10 nucleobase mutations.

In some aspects, the mutation count range is between and inclusive of 1-15 nucleobase changes or mutations, alternatively 1-14 nucleobase changes or mutations, alternatively 1-13 nucleobase changes or mutations, alternatively 1-12 nucleobase changes or mutations, alternatively 1-11 nucleobase changes or mutations, alternatively 1-10 nucleobase changes or mutations, alternatively 1-9 nucleobase changes or mutations, alternatively 1-8 nucleobase changes or mutations, alternatively 1-7 nucleobase changes or mutations, alternatively 1-6 nucleobase changes or mutations, alternatively 1-5 nucleobase changes or mutations, alternatively 1-4 nucleobase changes or mutations, alternatively 1-3 nucleobase changes or mutations.

In some aspects of the methods and systems disclosed herein, the range of GC content of a guide polynucleotide is between and inclusive of about 35% to about 65%, alternatively about 40% to about 60%, alternatively about 45% to about 55%, alternatively about 50% to about 55%.

In some aspects of the methods and systems disclosed herein, the maximum distance between a DNA break (e.g., single-strand cut, double-stand cut, or nick) and a site-specific modification in a target regulatory element nucleotide sequence is 80 bp, alternatively 75 bp, alternatively 70 bp, alternatively 65 bp, alternatively 60 bp, alternatively 55 bp, alternatively 50 bp, alternatively 45 bp, alternatively 40 bp, alternatively 35 bp, alternatively 30 bp, alternatively 25 bp, alternatively 20 bp, alternatively 15 bp, alternatively 10 bp.

In some aspects of the methods and systems disclosed herein, selecting a variant nucleotide includes more than one step of fitness score calculation, determination, or refinement. For example, in some aspects of the methods disclosed herein (i.e., artificial intelligence-mediated methods for editing a plant genome, artificial intelligence methods for predicting expression modifications in genetic variants, artificial intelligence-mediated methods for breeding genetically modified plants, and methods for editing a plant genome that include editing a plant genome to introduce one or more of site-specific edits), expression profile prediction and fitness score calculation includes (a) predicting one or more expression profiles for each variant nucleotide sequence relative to expression of the reference nucleotide sequence; (b) calculating an initial fitness score (e.g., a first fitness score) for each of the variant nucleotide sequences, wherein the fitness score reflects the degree to which a predicted expression profile for a variant nucleotide sequence meets a target expression profile, and wherein the fitness score incorporates one or more constraints that alter the suitability of the variant nucleotide sequence based on a genome editing system; (c) selecting a subset of variant nucleotide sequences based on the initial (first) fitness score for each variant nucleotide sequence; (d) providing the AI model with a third dataset, the third dataset comprising the subset of variant nucleotide sequences, wherein each variant nucleotide sequence of the subset of variant nucleotide sequences comprises an additional mutation or one or more mutations not found in the corresponding variant nucleotide sequences in the second dataset or results from a recombination of two or more of the variant nucleotide sequences from the second data set; (e) optionally repeating (a)-(d) until a variant nucleotide sequence of the subset of variant nucleotide sequences that meets a target fitness score is identified; and (f) selecting at least one final variant nucleotide sequence from the subset of variant nucleotide sequences, wherein the at least one final variant nucleotide sequence meets the target fitness score. As used herein, “recombination” and more specifically “recombination of two or more variant nucleotide sequences” refers to the exchange of nucleobases or a subset of nucleobases between a first variant nucleotide sequence and a second variant nucleotide sequence to derive a third nucleotide sequence having a portion or degree of sequence homology to both the first and second variant nucleotide sequences.

In some aspects of the methods and systems disclosed herein, the genome editing system comprises an endonuclease that introduces one or more site-specific modifications in the nucleotide sequence of one or more regulatory elements of a plant cell.

### Endonucleases

Endonucleases are enzymes that cleave the phosphodiester bond within a polynucleotide chain and include restriction endonucleases that cleave DNA at specific sites without damaging the bases. Site-specific modifications that are introduced with the disclosed methods and systems include those produced using double-stranded break technologies such as TAL effector nucleases, meganucleases, zinc finger nucleases, and Cas (CRISPR-associated) effector endonucleases.

Meganucleases have been classified into four families based on conserved sequence motifs. These motifs participate in the coordination of metal ions and hydrolysis of phosphodiester bonds. Meganucleases are notable for their long recognition sites, and for tolerating some sequence polymorphisms in their DNA substrates. TAL effector nucleases (TALENs) are a class of sequence-specific nucleases that are used to make double-strand breaks at specific target sequences in the genome of a plant or other organism. (Miller, et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site-specificity is conferred by the zinc finger domain, which typically comprising two, three, or four zinc fingers, for example having a C2H2 structure, however other zinc finger structures have been engineered. Zinc finger domains are amenable for designing polypeptides which specifically bind a selected polynucleotide recognition sequence. ZFNs include an engineered DNA-binding zinc finger domain linked to a nonspecific endonuclease domain, for example nuclease domain from a Type Ms endonuclease such as Fokl. Additional functionalities are fused to the zinc-finger binding domain, including transcriptional activator domains, transcription repressor domains, and methylases. In some examples, dimerization of a nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18-nucleotide recognition sequence.

In some aspects of the methods and systems disclosed herein, the genome editing system comprises a Cas endonuclease and one or more guide polynucleotides that introduce one or more site-specific modifications in the nucleotide sequence of one or more regulatory elements of a plant cell. For example, the methods and systems described herein can be used to introduce a CRISPR-Cas system into a plant cell or plant, for the purpose of genome modification of a target sequence (e.g., a plant regulatory element) in the genome of a plant or plant cell, for selecting plants, for deleting a base or a sequence, for gene editing, and for inserting a polynucleotide of interest into the genome of a plant or plant cell. Thus, the disclosed methods and systems can utilize a CRISPR-Cas system to provide for an effective system for modifying or altering target sites and nucleotides of interest within the genome of a plant cell or plant.

### CRISPR-Cas

CRISPR loci (Clustered Regularly Interspaced Short Palindromic Repeats) (also known as SPIDRs-SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

As used herein, “Cas protein”, “Cas polypeptide” refer to a polypeptide encoded by a Cas (CRISPR-associated) gene. Cas proteins or Cas polypeptides can be a “Cas endonuclease” or “Cas effector protein”, that when in complex with a suitable polynucleotide component, is capable of recognizing, binding to, and optionally nicking or cleaving all or part of a specific polynucleotide target sequence. A Cas polypeptide includes but is not limited to: Cas9, Cas12f (Cas-alpha, Cas14), Cas12l (Cas-beta), Cas12a (Cpf1), Cas12b (a C2c1 protein), Cas13 (a C2c2 protein), Cas12c (a C2c3 protein), Cas12d, Cas12e, Cas12g, Cas12h, Cas12i, Cas12j, Cas12k, Cas3, Cas3-HD, Cas 5, Cas6, Cas7, Cas8, Cas10, or combinations or complexes of these. In some aspects, the methods and compositions described herein can utilize transposon-associated TnpB, a programmable RNA-guided DNA endonuclease. A Cas endonuclease described herein can comprise one or more nuclease domains. The endonucleases of the disclosure may include those having one or more RuvC nuclease domains. Cas polypeptides further include functional fragments or functional variants of a native Cas polypeptide, or a protein that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity. As used herein, “functional fragment,” “fragment that is functionally equivalent,” and “functionally equivalent fragment” are used interchangeably and refer to a portion or sub-sequence of a Cas endonuclease sequence in which the ability to create a double-strand break is retained. As used herein, “functional variant,” “variant that is functionally equivalent”, and “functionally equivalent variant” are used interchangeably and refer to a variant of a Cas endonuclease in which the ability to create a double-strand break is retained. Fragments and variants are obtained via methods such as site-directed mutagenesis and synthetic construction.

As used herein, an “effector”, “effector protein”, or “effector polypeptide” is a polypeptide that encompasses an activity including recognizing, binding to, and/or cleaving or nicking a polynucleotide target. An effector, or effector protein, may also be an endonuclease. The “effector complex” of a CRISPR system includes Cas proteins involved in crRNA and target recognition and binding. Some of the component Cas proteins may additionally comprise domains involved in target polynucleotide cleavage.

Cas endonucleases, either as single effector proteins or in an effector complex with other components, unwind the DNA duplex at a target sequence and optionally cleave at least one DNA strand, as mediated by recognition of the target sequence by a polynucleotide (such as, but not limited to, a crRNA or guide RNA) that is in complex with the Cas endonuclease. Such recognition and cutting of a target sequence by a Cas endonuclease typically occurs if the correct protospacer-adjacent motif (PAM) is located at or adjacent to the 3′ end of the DNA target sequence. Alternatively, a Cas endonuclease herein may lack DNA cleavage or nicking activity, but can still specifically bind to a DNA target sequence when complexed with a suitable RNA component. (See also U.S. Patent Application US20150082478 published 19 Mar. 2015 and US20150059010 published 26 Feb. 2015).

Cas endonucleases of the methods and systems described herein include, but are not limited to, Cas3 (a feature of Class 1 type I systems), Cas9 (a feature of Class 2 type II systems),Cpf1 (a feature of Class 2 type V systems), and Cas-alpha.

Cas endonucleases and effector proteins can be used for targeted genome editing (via simplex and multiplex double-strand breaks and nicks) and targeted genome regulation (via tethering of epigenetic effector domains to either the Cas protein or sgRNA. A Cas endonuclease can also be engineered to function as an RNA-guided recombinase, and via RNA tethers could serve as a scaffold for the assembly of multiprotein and nucleic acid complexes (Mali et al., 2013, Nature Methods Vol. 10:957-963).

The Cas endonucleases described herein can be expressed and purified by methods known in the art, for example as described in WO/2016/186953 published 24 Nov. 2016.

The Cas endonuclease can comprise a modified form of the Cas polypeptide. The modified form of the Cas polypeptide can include an amino acid change (e.g., deletion, insertion, or substitution) that reduces the naturally-occurring nuclease activity of the Cas protein. For example, in some instances, the modified form of the Cas protein has less than 50%, less than 40%, less than 30%, less than 20%, less than 10%, less than 5%, or less than 1% of the nuclease activity of the corresponding wild-type Cas polypeptide (US20140068797 published 6 Mar. 2014). In some cases, the modified form of the Cas polypeptide has no substantial nuclease activity and is referred to as catalytically “inactivated Cas” or “deactivated Cas (dCas).” An inactivated Cas/deactivated Cas includes a deactivated Cas endonuclease (dCas). A catalytically inactive Cas endonuclease can be fused to a heterologous sequence to induce or modify activity.

A Cas endonuclease can be part of a fusion protein comprising one or more heterologous protein domains (e.g., 1, 2, 3, or more domains in addition to the Cas protein. Suitable fusion partners include, but are not limited to, a polypeptide that provides an activity that indirectly increases transcription by acting directly on the target DNA or on a polypeptide (e.g., a histone or other DNA-binding protein) associated with the target DNA. Additional suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. Further suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.). A catalytically inactive Cas can also be fused to a FokI nuclease to generate double-strand breaks (Guilinger et al. Nature Biotechnology, volume 32, number 6, June 2014). In some aspects, the Cas endonuclease is a fusion protein further comprising a nuclease domain, a transcriptional activator domain, a transcriptional repressor domain, an epigenetic modification domain, a cleavage domain, a nuclear localization signal, a cell-penetrating domain, a translocation domain, a marker, or a transgene that is heterologous to the target polynucleotide sequence or to the cell from which the target polynucleotide sequence is obtained or derived. In some aspects, the nuclease fusion protein comprises Clo51 or Fok1.

In some aspects of the methods and systems disclosed herein, a Cas endonuclease gene can be plant optimized, wherein the plant-optimized Cas endonuclease is capable of binding to and creating a double strand break in a genomic target sequence of a plant genome. As used herein, a “plant-optimized Cas endonuclease” (e.g., “plant optimized Cas9 endonuclease”, “plant optimized Cas-alpha endonuclease”, and “plant optimized Cas12f endonuclease”) refers to a Cas endonuclease encoded by a nucleotide sequence that has been optimized for expression in a plant cell or a plant. A “plant-optimized nucleotide sequence encoding a Cas endonuclease” and a “plant-optimized construct encoding a Cas endonuclease” are used interchangeably herein and refer to a nucleotide sequence encoding a Cas endonuclease polypeptide, or a variant or functional fragment thereof, that has been optimized for expression in a plant cell or plant. A plant comprising a plant-optimized Cas endonuclease includes a plant comprising the nucleotide sequence encoding for the Cas polypeptide sequence and/or a plant comprising the Cas endonuclease polypeptide. In some aspects, a plant-optimized Cas endonuclease nucleotide sequence results in increased Cas polypeptide expression when compared to the wild-type sequence of which it was optimized from. In some aspects, a plant-optimized nucleotide sequence encoding a Cas endonuclease can be a maize-optimized, canola-optimized, sunflower-optimized, rice-optimized, wheat-optimized, or soybean-optimized Cas endonuclease.

### Cas9 Endonuclease

In some aspects of the methods and systems disclosed herein, the genome editing system comprises a Cas9 endonuclease and one or more guide polynucleotides that introduce one or more site-specific modifications in the nucleotide sequence of one or more regulatory elements of a plant cell. In some aspects of the methods and systems disclosed herein, the genome editing system comprises a Cas9 endonuclease, one or more guide polynucleotides, and a donor DNA. Some exemplary Cas9 endonucleases are described, for example, in WO2019165168.

Cas9 (formerly referred to as Cas5, Csn1, or Csx12) is a Cas endonuclease that forms a complex with a crNucleotide and a tracrNucleotide, or with a single guide polynucleotide, for specifically recognizing and cleaving all or part of a DNA target sequence. The canonical Cas9 recognizes a 3′ GC-rich PAM sequence on a target dsDNA, typically comprising an NGG motif. The Cas endonucleases described herein may recognize additional PAM sequences and be used to modify target sites with different recognition sequence specificity.

A Cas9 protein comprises a RuvC nuclease with an HNH (H—N—H) nuclease adjacent to the RuvC-II domain. The RuvC nuclease and HNH nuclease each can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al., 2013, Cell 157:1262-1278). Cas9 endonucleases are typically derived from a type II CRISPR system, which includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA (Makarova et al. 2015, Nature Reviews Microbiology Vol. 13:1-15).

A Cas9 endonuclease, effector protein, or functional fragment thereof, for use in the disclosed methods and systems, can be isolated from a native source, or from a recombinant source where the genetically modified host cell is modified to express the nucleic acid sequence encoding the protein. Alternatively, the Cas endonuclease protein can be produced using cell free protein expression systems or be synthetically produced. Cas endonucleases can be isolated and introduced into a heterologous cell or can be modified from its native form to exhibit a different type or magnitude of activity than what it would exhibit in its native source. Such modifications include, but are not limited to, fragments, variants, substitutions, deletions, and insertions.

The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target. As used herein, the term “guide nucleotide” relates to a synthetic fusion of two RNA molecules, a crRNA (CRISPR RNA) comprising a variable targeting domain, and a tracrRNA. In an aspect, the guide nucleotide comprises a variable targeting domain of 12 to 30 nucleotide sequences and a RNA fragment that interacts with a Cas endonuclease.

### Cas-Alpha Endonuclease

In some aspects of the methods and systems disclosed herein, the genome editing system comprises a Cas-alpha (e.g., Cas12f) endonuclease and one or more guide polynucleotides that introduce one or more site-specific modifications in the nucleotide sequence of one or more regulatory elements of a plant cell. In some aspects of the methods and systems disclosed herein, the genome editing system comprises a Cas-alpha endonuclease, one or more guide polynucleotides, and a donor DNA. Some exemplary Cas-alpha endonucleases are described, for example, in U.S. Pat. No. 10,934,536 and WO2022082179.

A Cas-alpha endonuclease is a functional RNA-guided, PAM-dependent dsDNA cleavage protein of fewer than 800 amino acids, comprising: a C-terminal RuvC catalytic domain split into three subdomains and further comprising bridge-helix and one or more Zinc finger motif(s); and an N-terminal Rec subunit with a helical bundle, WED wedge-like (or “Oligonucleotide Binding Domain”, OBD) domain, and, optionally, a Zinc finger motif.

Cas-alpha endonucleases comprise one or more Zinc Finger (ZFN) coordination motif(s) that may form a Zinc binding domain. Zinc Finger-like motifs can aid in target and non-target strand separation and loading of the guide RNA into the DNA target. Cas-alpha endonucleases comprising one or more Zinc Finger motifs can provide additional stability to a ribonucleoprotein complex on a target polynucleotide. Cas-alpha endonucleases comprise C4 or C3H zinc binding domains.

A Cas-alpha endonuclease can function as a double-strand-break-inducing agent, a single-strand-break inducing agent, or as a nickase. In some aspects, a catalytically inactive Cas-alpha endonuclease can be used to target or recruit to a target DNA sequence but not induce cleavage. In some aspects, a catalytically inactive Cas-alpha protein can be combined with a base editing molecule, such as a cytidine deaminase or an adenine deaminase.

A Cas-alpha endonuclease, effector protein, or functional fragment thereof, can be used in the disclosed methods and systems for targeted genome editing (via simplex and multiplex double-strand breaks and nicks). In some aspects of the methods and systems disclosed herein, a genome editing system comprises Cas12f.

### Guide Polynucleotides

A guide polynucleotide enables target recognition, binding, and optionally cleavage by the Cas endonuclease, and can be a single molecule or a double molecule. The guide polynucleotide sequence can be a RNA sequence, a DNA sequence, or a combination thereof (a RNA-DNA combination sequence). As used herein, “guide polynucleotide/Cas endonuclease complex”, “guide polynucleotide/Cas endonuclease system”, “guide polynucleotide/Cas complex”, “guide polynucleotide/Cas system” and “guided Cas system” are used interchangeably and refer to at least one guide polynucleotide and at least one Cas endonuclease, that are capable of forming a complex, wherein the guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems (Horvath and Barrangou, 2010, Science 327:167-170; Makarova et al. 2015, Nature Reviews Microbiology Vol. 13:1-15; Zetsche et al., 2015, Cell 163, 1-13; Shmakov et al., 2015, Molecular Cell 60, 1-13).

As used herein, “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein the guide RNA/Cas endonuclease complex can direct the Cas endonuclease to a DNA target site, enabling the Cas endonuclease to recognize, bind to, and optionally nick or cleave (introduce a single or double-strand break) the DNA target site.

Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA”. A guide polynucleotide may be engineered or synthetic.

The guide polynucleotide can include a chimeric non-naturally occurring guide polynucleotide comprising regions that are not found together in nature (i.e., they are heterologous with respect to each other). For example, a chimeric non-naturally occurring guide polynucleotide comprising a first nucleotide sequence domain (referred to as Variable Targeting domain or VT domain) that can hybridize to a nucleotide sequence in a target DNA, linked to a second nucleotide sequence that can recognize the Cas endonuclease, such that the first and second nucleotide sequence are not found linked together in nature.

In some aspects, the RNA that guides the RNA/Cas endonuclease complex is a duplexed RNA comprising a duplex crRNA-tracrRNA. The guide polynucleotide can be a double molecule (also referred to as duplex guide polynucleotide) comprising a crNucleotide sequence and a tracrNucleotide sequence. The crNucleotide includes a first nucleotide sequence domain (referred to as Variable Targeting domain or VT domain) that can hybridize to a nucleotide sequence in a target DNA and a second nucleotide sequence (also referred to as a tracr mate sequence) that is part of a Cas endonuclease recognition (CER) domain. The tracr mate sequence can hybridized to a tracrNucleotide along a region of complementarity and together form the Cas endonuclease recognition domain or CER domain. The CER domain is capable of interacting with a Cas endonuclease polypeptide. The crNucleotide and the tracrNucleotide of the duplex guide polynucleotide can be RNA, DNA, and/or RNA-DNA-combination sequences.

In some aspects, the crNucleotide molecule of the duplex guide polynucleotide is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the crRNA naturally occurring in Bacteria and Archaea. The size of the fragment of the crRNA naturally occurring in Bacteria and Archaea that can be present in a crNucleotide disclosed herein can range from, but is not limited to, 2, 3, 4, 5, 6, 7, 8, 9, 10, 11, 12, 13, 14, 15, 16, 17, 18, 19, 20 or more nucleotides.

The tracrRNA (trans-activating CRISPR RNA) comprises, in the 5′-to-3′ direction, (i) an “anti-repeat” sequence that anneals with the repeat region of CRISPR type II crRNA and (ii) a stem loop-comprising portion (Deltcheva et al., Nature 471:602-607). The duplex guide polynucleotide can form a complex with a Cas endonuclease, wherein the guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double-strand break) into the target site. (US20150082478 published 19 Mar. 2015 and US20150059010 published 26 Feb. 2015). In some aspects, the tracrNucleotide is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides.

Nucleotide sequence modifications of the guide polynucleotide, VT domain, and/or CER domain is selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide.

The VT domain and/or the CER domain of a single guide polynucleotide can comprise a RNA sequence, a DNA sequence, or a RNA-DNA-combination sequence. The single guide polynucleotide being comprised of sequences from the crNucleotide and the tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas endonuclease, wherein the guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double-strand break) the target site. (US20150082478 published 19 Mar. 2015 and US20150059010 published 26 Feb. 2015).

A chimeric non-naturally occurring single guide RNA (sgRNA) includes a sgRNA that comprises regions that are not found together in nature (i.e., they are heterologous with each other. For example, a sgRNA comprising a first nucleotide sequence domain (referred to as Variable Targeting domain or VT domain) that can hybridize to a nucleotide sequence in a target DNA linked to a second nucleotide sequence (also referred to as a tracr mate sequence) that are not found linked together in nature.

The nucleotide sequence linking the crNucleotide and the tracrNucleotide of a single guide polynucleotide can comprise a RNA sequence, a DNA sequence, or a RNA-DNA combination sequence. In some aspects, the nucleotide sequence linking the crNucleotide and the tracrNucleotide of a single guide polynucleotide (also referred to as “loop”) can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In some aspects, the nucleotide sequence linking the crNucleotide and the tracrNucleotide of a single guide polynucleotide can comprise a tetraloop sequence, such as, but not limiting to a GAAA tetraloop sequence.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas9 system that can form a complex with a type II Cas9 endonuclease, wherein the guide RNA/Cas9 endonuclease complex can direct the Cas9 endonuclease to a DNA target site, enabling the Cas9 endonuclease to recognize, bind to, and optionally nick or cleave (introduce a single or double strand break) the DNA target site.

Single guide RNAs targeting a target site in the genome of an organism can be designed by changing the Variable Targeting Domain (VT) of any of the guide polynucleotides described herein, with any random nucleotide that can hybridize to any desired target sequence.

In some aspects, a subject nucleic acid (e.g., a guide polynucleotide, a nucleic acid comprising a nucleotide sequence encoding a guide polynucleotide; a nucleic acid encoding Cas9 endonuclease of the present disclosure; a crRNA or a nucleotide encoding a crRNA, a tracrRNA or a nucleotide encoding a tracrRNA, a nucleotide encoding a VT domain, a nucleotide encoding a CER domain, etc.) comprises a modification or sequence that provides for an additional desirable feature (e.g., modified or regulated stability; subcellular targeting; tracking, e.g., a fluorescent label; a binding site for a protein or protein complex; etc.). Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6 -Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

### Protospacer Adjacent Motif (PAM)

A “protospacer adjacent motif” (PAM) herein refers to a short nucleotide sequence adjacent to a target sequence (protospacer) that can be recognized (targeted) by a guide polynucleotide/Cas endonuclease system. In some aspects, the Cas endonuclease may not successfully recognize a target DNA sequence if the target DNA sequence is not adjacent to, or near, a PAM sequence. In some aspects, the PAM precedes the target sequence (e.g., Cas12a). In some aspects, the PAM follows the target sequence (e.g., S. pyogenes Cas9). The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

A “randomized PAM” and “randomized protospacer adjacent motif” are used interchangeably herein, and refer to a random DNA sequence adjacent to a target sequence (protospacer) that is recognized (targeted) by a guide polynucleotide/Cas endonuclease system. The randomized PAM sequence can be of any length but is typically 1, 2, 3, 4, 5, 6, 7, 8, 9, 10, 11, 12, 13, 14, 15, 16, 17, 18, 19 or 20 nucleotides long. A randomized nucleotide includes anyone of the nucleotides A, C, G or T.

Many Cas endonucleases have been described to date that can recognize specific PAM sequences (WO2016186953 published 24 Nov. 2016, WO2016186946 published 24 Nov. 2016, and Zetsche B et al. 2015. Cell 163, 1013) and cleave the target DNA at a specific position.

### Guide Polynucleotide/Cas Endonuclease Complexes

The guide polynucleotide/Cas endonuclease complexes for the methods and systems described herein are capable of recognizing, binding to, and optionally nicking, unwinding, or cleaving all or part of a target sequence.

A guide polynucleotide/Cas endonuclease complex that can cleave both strands of a DNA target sequence typically comprises a Cas protein that has all of its endonuclease domains in a functional state (e.g., wild-type endonuclease domains or variants thereof retaining some or all activity in each endonuclease domain). Thus, a wild-type Cas polypeptide (e.g., a Cas polypeptide disclosed herein), or a variant thereof retaining some or all activity in each endonuclease domain of the Cas polypeptide, is a suitable example of a Cas endonuclease that can cleave both strands of a DNA target sequence.

A guide polynucleotide/Cas endonuclease complex that can cleave one strand of a DNA target sequence can be characterized herein as having nickase activity (e.g., partial cleaving capability). A Cas nickase typically comprises one functional endonuclease domain that allows the Cas to cleave only one strand (i.e., make a nick) of a DNA target sequence. For example, a Cas nickase may comprise (i) a mutant, dysfunctional RuvC domain and (ii) a functional HNH domain (e.g., wild-type HNH domain). As another example, a Cas nickase may comprise (i) a functional RuvC domain (e.g., wild-type RuvC domain) and (ii) a mutant, dysfunctional HNH domain. Non-limiting examples of Cas nickases suitable for use herein are disclosed in US20140189896 published on 3 Jul. 2014. A pair of Cas nickases can be used to increase the specificity of DNA targeting. In general, this can be done by providing two Cas nickases that, by virtue of being associated with RNA components with different guide sequences, target and nick nearby DNA sequences on opposite strands in the region for desired targeting. Such nearby cleavage of each DNA strand creates a double-strand break (i.e., a DSB with single-stranded overhangs), which is then recognized as a substrate for non-homologous-end-joining, NHEJ (prone to imperfect repair leading to mutations) or homologous recombination, HR. Each nick can be at least 5, between 5 and 10, at least 10, between 10 and 15, at least 15, between 15 and 20, at least 20, between 20 and 30, at least 30, between 30 and 40, at least 40, between 40 and 50, at least 50, between 50 and 60, at least 60, between 60 and 70, at least 70, between 70 and 80, at least 80, between 80 and 90, at least 90, between 90 and 100, or 100 or greater (or any number between 5 and 100) bases apart from each other, for example.

A guide polynucleotide/Cas endonuclease complex can bind to a DNA target site sequence, but does not cleave any strand at the target site sequence. Such a complex may comprise a Cas protein in which all of its nuclease domains are mutant, dysfunctional. For example, a Cas protein that can bind to a DNA target site sequence, but does not cleave any strand at the target site sequence, may comprise both a mutant, dysfunctional RuvC domain and a mutant, dysfunctional HNH domain. A Cas protein herein that binds, but does not cleave, a target DNA sequence can be used to modulate gene expression, for example, in which case the Cas protein could be fused with a transcription factor (or portion thereof) (e.g., a repressor or activator, such as any of those disclosed herein).

In some aspects of the disclosure, the guide polynucleotide/Cas endonuclease complex is a guide polynucleotide/Cas endonuclease complex (PGEN) comprising at least one guide polynucleotide and at least one Cas endonuclease polypeptide. In some aspects, the Cas endonuclease polypeptide comprises at least one protein subunit of another Cas protein, or a functional fragment thereof, wherein the guide polynucleotide is a chimeric non-naturally occurring guide polynucleotide, wherein the guide polynucleotide/Cas endonuclease complex is capable of recognizing, binding to, and optionally nicking, unwinding, or cleaving all or part of a target sequence.

In some aspects, the guide polynucleotide/Cas endonuclease complex can be a ribonucleoprotein complex (RNP), wherein the Cas endonuclease is provided as a protein and the guide polynucleotide is provided as a ribonucleotide.

In some aspects of the disclosure, the guide polynucleotide/Cas effector complex is a guide polynucleotide/Cas endonuclease complex comprising at least one guide polynucleotide and a Cas endonuclease, wherein the guide polynucleotide/Cas endonuclease complex is capable of recognizing, binding to, and optionally nicking, unwinding, or cleaving all or part of a target sequence.

The PGEN can be a guide polynucleotide/Cas endonuclease complex, wherein the Cas endonuclease further comprises one copy or multiple copies of at least one protein subunit, or a functional fragment thereof, of an additional Cas protein.

Any component of the guide polynucleotide/Cas endonuclease complex, the guide polynucleotide/Cas endonuclease complex itself, as well as the polynucleotide modification template(s) and/or donor DNA(s), can be introduced into a heterologous cell or organism by any method known in the art.

Some uses for guide polynucleotide/Cas endonuclease systems include but are not limited to modifying or replacing nucleotide sequences of interest (such as a regulatory elements), insertion of polynucleotides of interest, genetic knock-out, genetic knock-in, modification of splicing sites and/or introducing alternate splicing sites, modifications of nucleotide sequences encoding a protein of interest, amino acid and/or protein fusions, and gene silencing by expressing an inverted repeat into a gene of interest.

As used herein, “knock-out” and “genetic knockout” are used interchangeably and refer to a DNA sequence that has been rendered partially or completely inoperative by targeting with the methods and systems described herein. A “knock-in” or “genetic knock-in” both refer to the replacement or insertion of a DNA sequence at a specific DNA integration point in the genome of a cell using the methods and systems described herein.

### NHEJ and HR

In some aspects of the methods and systems described herein, the genome editing system comprises a Cas endonuclease, one or more guide polynucleotides, and optionally donor DNA, and editing a target regulatory element nucleotide sequence comprises nonhomologous end-joining (NHEJ) or homologous recombination (HR) following a Cas endonuclease-mediated double-strand break. Once a double-strand break is induced in the DNA, the cell's DNA repair mechanism is activated to repair the break. The most common repair mechanism to bring the broken ends together is the nonhomologous end-joining pathway (Bleuyard et al., (2006) DNA Repair 5:1-12). The structural integrity of chromosomes is typically preserved by the repair, but deletions, insertions, or other rearrangements are possible (Siebert and Puchta, (2002) Plant Cell 14:1121-31; Pacher et al., (2007) Genetics 175:21-9). Alternatively, the double-strand break can be repaired by homologous recombination between homologous DNA sequences. Once the sequence around the double-strand break is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

In some aspects of the methods and systems described herein, the genome editing system comprises a Cas endonuclease, one or more guide polynucleotides, and a donor DNA. As used herein, “donor DNA” is a DNA construct that comprises a polynucleotide of interest to be inserted into the target site of a Cas endonuclease. Once a double-strand break is introduced in the target site by the endonuclease, the first and second regions of homology of the donor DNA can undergo homologous recombination with their corresponding genomic regions of homology resulting in exchange of DNA between the donor and the target genome. As such, the provided methods result in the integration of the polynucleotide of interest of the donor DNA into the double-strand break in the target site in the plant genome, thereby altering the original target site and producing an altered genomic target site.

In some aspects of the methods and systems described herein, the genome editing system comprises a Cas endonuclease, one or more guide polynucleotides, and optionally donor DNA, and editing a plant genome comprises editing a target regulatory element nucleotide sequence in a plant cell such that the target regulatory element nucleotide sequence aligns with a final selected variant nucleotide sequence.

In some aspects of the methods and systems described herein, the genome editing system comprises a Cas-alpha endonuclease, one or more guide polynucleotides, and optionally donor DNA, and editing a plant genome comprises editing a target regulatory element nucleotide sequence in a plant cell such that the target regulatory element nucleotide sequence aligns with a final selected variant nucleotide sequence.

In some aspects of the methods and systems described herein, the genome editing system comprises a Cas12f endonuclease, one or more guide polynucleotides, and optionally donor DNA, and editing a plant genome comprises editing a target regulatory element nucleotide sequence in a plant cell such that the target regulatory element nucleotide sequence aligns with a final selected variant nucleotide sequence.

In some aspects of the methods and systems described herein, the genome editing system comprises a Cas9 endonuclease, one or more guide polynucleotides, and optionally donor DNA, and editing a plant genome comprises editing a target regulatory element nucleotide sequence in a plant cell such that the target regulatory element nucleotide sequence aligns with a final selected variant nucleotide sequence.

In some aspects of the methods and systems described herein, the genome editing system comprises a Cas endonuclease, one or more guide polynucleotides, and optionally donor DNA, and editing a target regulatory element nucleotide sequence comprises introducing at least one site-specific modification in a target regulatory element nucleotide sequence (e.g., at least one nucleotide insertion, at least one nucleotide deletion, at least one nucleotide substitution, or a combination thereof) to achieve a selected variant nucleotide sequence.

In some aspects of the methods and systems described herein, the genome editing system comprises a Cas-alpha endonuclease, one or more guide polynucleotides, and optionally donor DNA, and editing a target regulatory element nucleotide sequence comprises introducing at least one site-specific modification in a target regulatory element nucleotide sequence (e.g., at least one nucleotide insertion, at least one nucleotide deletion, at least one nucleotide substitution, or a combination thereof) to achieve a selected variant nucleotide sequence.

In some aspects of the methods and systems described herein, the genome editing system comprises a Cas12f endonuclease, one or more guide polynucleotides, and optionally donor DNA, and editing a target regulatory element nucleotide sequence comprises introducing at least one site-specific modification in a target regulatory element nucleotide sequence (e.g., at least one nucleotide insertion, at least one nucleotide deletion, at least one nucleotide substitution, or a combination thereof) to achieve a selected variant nucleotide sequence.

In some aspects of the methods and systems described herein, the genome editing system comprises a Cas9 endonuclease, one or more guide polynucleotides, and optionally donor DNA, and editing a target regulatory element nucleotide sequence comprises introducing at least one site-specific modification in a target regulatory element nucleotide sequence (e.g., at least one nucleotide insertion, at least one nucleotide deletion, at least one nucleotide substitution, or a combination thereof) to achieve a selected variant nucleotide sequence.

### Base Editing

In some aspects of the methods and systems described herein, the genome editing system comprises a base editing agent and a plurality of guide polynucleotides and editing a target regulatory element nucleotide sequence comprises introducing a plurality of nucleobase edits in the target regulatory element nucleotide sequence resulting in a variant nucleotide sequence.

One or more nucleobases of a target polynucleotide can be chemically altered, in some cases to change the base from one type to another, for example from a Cytosine to a Thymine, or an Adenine to a Guanine. In some aspects, a plurality of bases, for example 2 or more, 5 or more, 10 or more, 20 or more, 30 or more, 40 or more, 50 or more, 60 or more, 70 or more, 80 or more 90 or more, 100 or more, or even greater than 100, 200 or more, up to thousands of bases may be modified or altered, to produce a plant with a plurality of modified bases.

Any base editing complex, such as a base editing agent associated with an RNA-guided polypeptide, can be used to target and bind to a desired locus in the genome of an organism and chemically modify one or more components of a target polynucleotide.

Site-specific base conversions can be achieved to engineer one or more nucleotide changes to create one or more edits into the genome. These include for example, a site-specific base edit mediated by an C·G to T·A or an A·T to G·C base editing deaminase enzymes (Gaudelli et al., Programmable base editing of A·T to G·C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016): 420-4). A catalytically “dead” or inactive Cas (dCas) endonuclease, for example a catalytically inactive “dead” version of a Cas endonuclease disclosed herein, fused to a cytidine deaminase or an adenine deaminase protein becomes a specific base editor that can alter DNA bases without inducing a DNA break. Base editors convert C->T (or G->A on the opposite strand) or an adenine base editor that would convert adenine to inosine, resulting in an A->G change within an editing window specified by the guide polynucleotide. As used herein, a “base editing agent” refers to a molecule that effects a change in a nucleobase.

For many traits of interest, the creation of double-strand breaks and the subsequent repair via HDR or NHEJ is not ideal for quantitative traits. An observed phenotype includes both genotype effects and environmental effects. The genotype effects further comprise additive effects, dominance effects, and epistatic effects. The probability of no effect per any single edit can be greater than zero, and any single phenotypic effect can be small, depending on the method used and site selected. Double-stranded break repair can additionally be “noisy” and have low repeatability.

One approach to ameliorate the probability of no effect per edit or small phenotypic effect outcome is to multiplex genome modification, such that a plurality of target sites are modified. Methods to modify a genomic sequence that do not introduce double-strand breaks would allow for single base substitutions. Combining these approaches, multiplexed base editing is beneficial for creating large numbers of genotype edits that can produce observable phenotype modifications. In some cases, dozens or hundreds or thousands of sites can be edited within one or a few generations of an organism.

A multiplexed approach to base editing in a plant, has the potential to create a plurality of significant phenotypic variations in one or a few generations, with a positive directional bias to the effects. A plant or a population of plants with a plurality of edits can be cross-bred to produce progeny plants, some of which will comprise multiple pluralities of edits from the parental lines. In this way, accelerated breeding of desired traits can be accomplished in parallel in one or a few generations, replacing time-consuming traditional sequential crossing and breeding across multiple generations.

A “deaminase” is an enzyme that catalyzes a deamination reaction. For example, deamination of adenine with an adenine deaminase results in the formation of inosine. Inosine selectively base pairs with cytosine instead of thymine. This results in a post-replicative transition mutation, such that the original A-T base pair transforms into a G-C base pair. In another example, cytosine deamination results in the formation of uracil, which can be repaired by cellular repair mechanisms back to a C-T base pair or to a T-A, G-C, or A-T base pair. This heterogeneity in repair can be suppressed by the introduction of a uracil glycosylase inhibitor, such that DNA repair or replication transforms the original C-T base pair into a T-A base pair (Burnett et al. (2022) Frontiers in Genome Editing. 4, 923718). In the case of both adenine and cytosine deaminases, the introduction of a nick promotes the respective base pair change (Burnett et al., 2022).

As used herein, a “dead” or “deactivated” Cas endonuclease or polypeptide (dCas) has been modified to lack the capability for creating either a single- or double-strand break in a target polynucleotide. A nickase Cas protein (nCas) has been modified to lack the capability for creating a double-strand break in a target double-stranded polynucleotide but retains the capability for cleaving or nicking one strand of a double-stranded polynucleotide.

A base editing deaminase, such as a cytidine deaminase or an adenine deaminase, may be fused to an RNA-guided endonuclease that can be deactivated (“dCas”, such as a deactivated Cas9) or partially active (“nCas”, such as a Cas9 nickase) so that it does not cleave a target site to which it is guided. The dCas forms a functional complex with a guide polynucleotide that shares homology with a polynucleotide sequence at the target site, and is further complexed with the deaminase molecule. The guided Cas endonuclease recognizes and binds to a double-stranded target sequence, opening the double-strand to expose individual bases. In the case of a cytidine deaminase, the deaminase deaminates the cytosine base and creates a uracil. Uracil glycosylase inhibitor (UGI) is provided to prevent the conversion of U back to C. DNA replication or repair mechanisms then convert the Uracil to a thymine (U to T), and subsequent repair of the opposing base (formerly G in the original G-C pair) to an Adenine, creating a T-A pair. For example, see Komor et al. Nature Volume 533, Pages 420-424, 19 May 2016.

In some aspects of the methods and systems described herein, the genome editing system comprises a base editing agent and a plurality of guide polynucleotides, and editing a plant genome comprises editing a target regulatory element nucleotide sequence in a plant cell to introduce a plurality of nucleobase edits in the target regulatory element nucleotide sequence resulting in a selected variant nucleotide sequence. In some aspects, the plurality of nucleobase edits is at least 10 site-specific nucleobase edits, alternatively at least 100 site-specific nucleobase edits, alternatively at least site-specific 1000 nucleobase edits.

In some aspects of the methods and systems described herein, the genome editing system comprises dCas-alpha complexed to a cytosine deaminase or an adenosine deaminase and a plurality of guide polynucleotides, and editing a plant genome comprises editing a target regulatory element nucleotide sequence in a plant cell to introduce a plurality of nucleobase edits in the target regulatory element nucleotide sequence resulting in a selected variant nucleotide sequence. In some aspects, the plurality of nucleobase edits is at least 10 site-specific nucleobase edits, alternatively at least 100 site-specific nucleobase edits, alternatively at least site-specific 1000 nucleobase edits.

In some aspects of the methods and systems described herein, the genome editing system comprises dCas12f complexed to a cytosine deaminase or an adenosine deaminase and a plurality of guide polynucleotides, and editing a plant genome comprises editing a target regulatory element nucleotide sequence in a plant cell to introduce a plurality of nucleobase edits in the target regulatory element nucleotide sequence resulting in a selected variant nucleotide sequence. In some aspects, the plurality of nucleobase edits is at least 10 site-specific nucleobase edits, alternatively at least 100 site-specific nucleobase edits, alternatively at least site-specific 1000 nucleobase edits.

In some aspects of the methods and systems described herein, the genome editing system comprises dCas9 complexed to a cytosine deaminase or an adenosine deaminase and a plurality of guide polynucleotides, and editing a plant genome comprises editing a target regulatory element nucleotide sequence in a plant cell to introduce a plurality of nucleobase edits in the target regulatory element nucleotide sequence resulting in a selected variant nucleotide sequence. In some aspects, the plurality of nucleobase edits is at least 10 site-specific nucleobase edits, alternatively at least 100 site-specific nucleobase edits, alternatively at least site-specific 1000 nucleobase edits.

In some aspects of the methods and systems described herein, the genome editing system comprises a base editing agent and a plurality of guide polynucleotides, and editing a plant genome comprises editing a target regulatory element nucleotide sequence comprises multiplex base editing with the base editing agent and the plurality of guide polynucleotides. In some aspects, multiplex base editing introduces at least 10 site-specific nucleobase edits, alternatively at least 100 site-specific nucleobase edits, alternatively at least site-specific 1000 nucleobase edits.

In some aspects of the methods and systems described herein, the genome editing system comprises dCas-alpha complexed to a cytosine deaminase or an adenosine deaminase and a plurality of guide polynucleotides, and editing a plant genome comprises editing a target regulatory element nucleotide sequence comprises multiplex base editing with the base editing agent and the plurality of guide polynucleotides. In some aspects, multiplex base editing introduces at least 10 site-specific nucleobase edits, alternatively at least 100 site-specific nucleobase edits, alternatively at least site-specific 1000 nucleobase edits.

In some aspects of the methods and systems described herein, the genome editing system comprises dCas12f complexed to a cytosine deaminase or an adenosine deaminase and a plurality of guide polynucleotides, and editing a plant genome comprises editing a target regulatory element nucleotide sequence comprises multiplex base editing with the base editing agent and the plurality of guide polynucleotides. In some aspects, multiplex base editing introduces at least 10 site-specific nucleobase edits, alternatively at least 100 site-specific nucleobase edits, alternatively at least site-specific 1000 nucleobase edits.

In some aspects of the methods and systems described herein, the genome editing system comprises dCas9 complexed to a cytosine deaminase or an adenosine deaminase and a plurality of guide polynucleotides, and editing a plant genome comprises editing a target regulatory element nucleotide sequence comprises multiplex base editing with the base editing agent and the plurality of guide polynucleotides. In some aspects, multiplex base editing introduces at least 10 site-specific nucleobase edits, alternatively at least 100 site-specific nucleobase edits, alternatively at least site-specific 1000 nucleobase edits.

### Prime Editing

In some aspects of the methods and systems described herein, the genome editing system comprises a prime editing agent and a guide polynucleotide and editing a target regulatory element nucleotide sequence comprises introducing one or more insertions, deletions, or nucleobase swaps in a target regulatory element nucleotide sequence without generating a double-stranded DNA break.

In some aspects, the prime editing agent is a Cas polypeptide fused to a reverse transcriptase, wherein the Cas polypeptide is modified to nick DNA rather than generating double-strand break. This Cas-polypeptide-reverse transcriptase fusion can also be referred to as a “prime editor” or “PE”. In some aspects, the guide polynucleotide comprises a prime editing guide RNA (pegRNA), and is larger than standard sgRNAs commonly used for CRISPR gene editing (e.g., >100 nucleobases). The pegRNA comprises a primer binding sequence (PBS) and a template containing the desired or target RNA sequence at its 3′ end.

During prime editing, the PE: pegRNA complex binds to a target DNA sequence and the modified Cas polypeptide nicks one target DNA strand resulting in a flap. The PBS on the pegRNA binds to the DNA flap and the target RNA sequence is reverse transcribed using the reverse transcriptase. The edited strand is incorporated into the target DNA at the end of the nicked flap, and the target DNA sequence is repaired with the new reverse transcribed DNA.

In some aspects of the methods and systems described herein, the genome editing system comprises a catalytically inactive Cas-alpha polypeptide (e.g., a Cas-alpha nickase) complexed or fused to a reverse transcriptase and a pegRNA, and editing a plant genome comprises editing a target regulatory element nucleotide sequence via prime editing.

In some aspects of the methods and systems described herein, the genome editing system comprises a catalytically inactive Cas12f polypeptide (e.g., a Cas12f nickase) complexed or fused to a reverse transcriptase and a pegRNA, and editing a plant genome comprises editing a target regulatory element nucleotide sequence via prime editing.

In some aspects of the methods and systems described herein, the genome editing system comprises a catalytically inactive Cas9 polypeptide (e.g., a Cas9 nickase) complexed or fused to a reverse transcriptase and a pegRNA, and editing a plant genome comprises editing a target regulatory element nucleotide sequence via prime editing.

All patents, publications, and patent applications mentioned in the specification are indicative of the level of those skilled in the art to which this disclosure pertains. All patents, publications, and patent applications are herein incorporated by reference in the entirety to the same extent as if each individual patent, publication, or patent application was specifically and individually indicated to be incorporated by reference in its entirety. The following examples are offered by way of illustration and not by way of limitation.

### EXAMPLES

The aspects of the disclosure are further defined in the following Examples, in which parts and percentages are by weight and degrees are Celsius, unless otherwise stated. These Examples, while indicating aspects of the disclosure, are given by way of illustration only. From the above discussion and these Examples, one skilled in the art can ascertain the essential characteristics of the aspects of the disclosure, and without departing from the spirit and scope thereof, can make various changes and modifications of them to adapt to various usages and conditions. Thus, various modifications in addition to those shown and described herein will be apparent to those skilled in the art from the foregoing description. Such modifications are also intended to fall within the scope of the appended claims.

### Example 1: Training and Validation of a Deep Neural Network

To develop an angiosperm-specific, transformer-based backbone for a polynucleotide prediction task, a BIG BIRD deep learning model [Zaheer et al., “Big Bird: Transformers for Longer Sequences, Neural Information Processing Systems (NeurIPS) 2020, arXiv: 2007.14062, doi.org/10.48550/arXiv.2007.14062; incorporated by reference] was provided genomes from various monocot and dicot species for self-supervised pretraining.

Prior to pretraining and fine-tuning, maize genes were divided into “fine-tune training”, “fine-tune validation”, and “fine-tune testing” sets. The fine-tune validation set consisted of genes in the first half of maize chromosome 9, and the fine-tune testing set consisted of genes in the first half of maize chromosome 10. For subsequent fine-tune training, to ensure testing performance would reflect generalization to new polynucleotide sequences, syntenic paralogs to genes in the fine-tune testing set were removed from the fine-tune training and validation sets based on syntenic region annotation in B73 maize. Additionally, maize chromosomes 9 and 10 were held for validation and testing, respectively, during the pretraining phase.

The pretraining species (“pretraining genomes”) included Gossypium raimondii, Brassica rapa, Medicago truncatula, Setaria italica, Panicum hallii, Solanum lycopersicum, Zea mays, Hordeum vulgare, Oryza sativa, Glycine max, Musa acuminata, Sorghum bicolor, Helianthus annuus, Triticum aestivum, and Arabidopsis thaliana. One chromosome from each species was retained for validation (“pretraining validation set)—monitoring held out pretraining task performance during pretraining, while a second chromosome from each species was held out for final testing (“pretraining species testing set”). All other chromosomes in each species were sampled as part of the pretraining task.

### Pretraining

Pretraining occurred in four stages, each stage having 200 epochs with a batch size of 256. The stages denote the maximum number of k-mers sampled for any single sequence, which initiated at 128 (Stage 1) and then increased to 256 (Stage 2), 512 (Stage 3), and finally 1024 k-mers (Stage 4). Polynucleotide sequences having lengths between 160 and 5,120 base pairs (bp) (hereinafter “pretraining sequences”) were randomly selected from across the pretraining genome chromosomes not held out for validation and testing. Pretraining sequences were encoded as input for the BIG BIRD deep learning model as a set of non-overlapping 5-mers, such that the token counts of the dataset ranged from 32 to 1024. Pretraining was based on a Masked Language Model (MLM) task, wherein the objective of the task was to infer, deduce, and identify missing or incorrect tokens based on the surrounding sequence context.

Within the pretraining dataset, 15% of tokens were randomly selected for masking. Of the tokens selected for masking, 80% of these tokens were masked with a special “MASK” token, 10% were masked with a token having a randomly assigned incorrect k-mer, and 10% of the “masked” tokens retained their original token identity. Following each epoch of pretraining, masked prediction was conducted on the pretraining validation set in order to monitor held-out sequence performance during training. The pretraining testing sequences were held out until the completion of the pretraining process, in order to assess generalization to completely held out sequences.

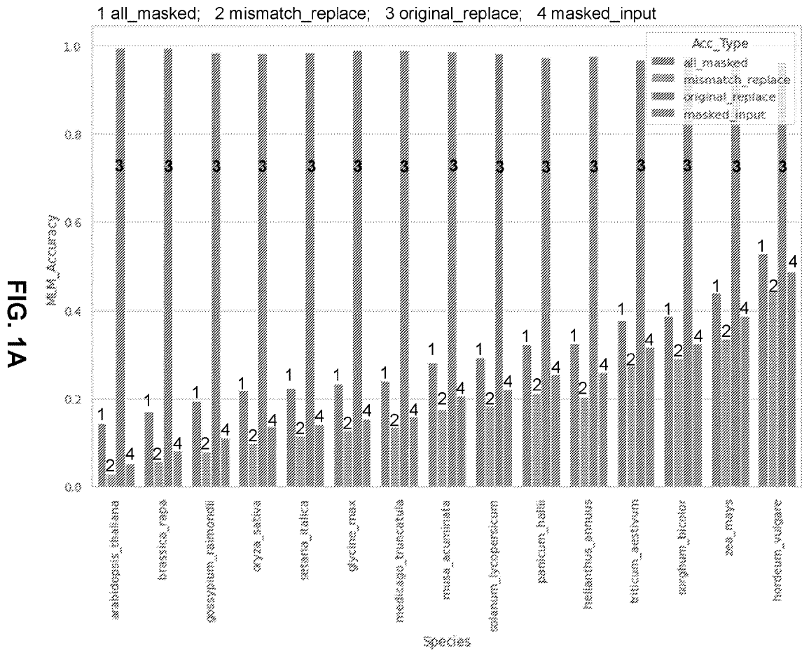

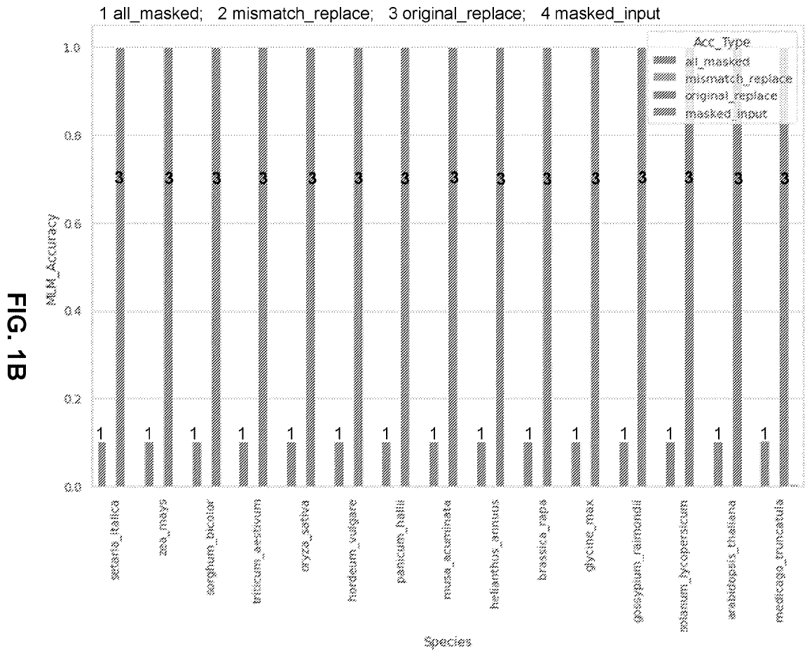

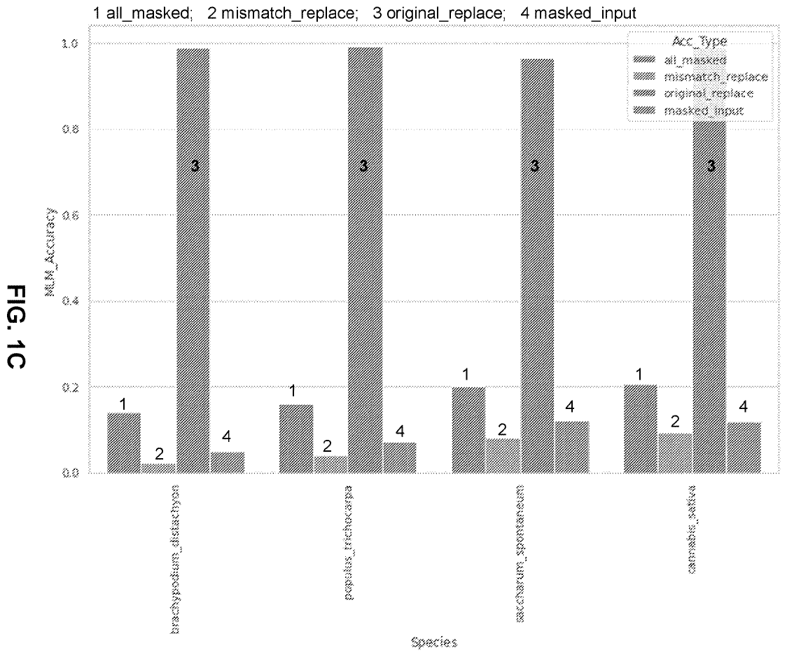

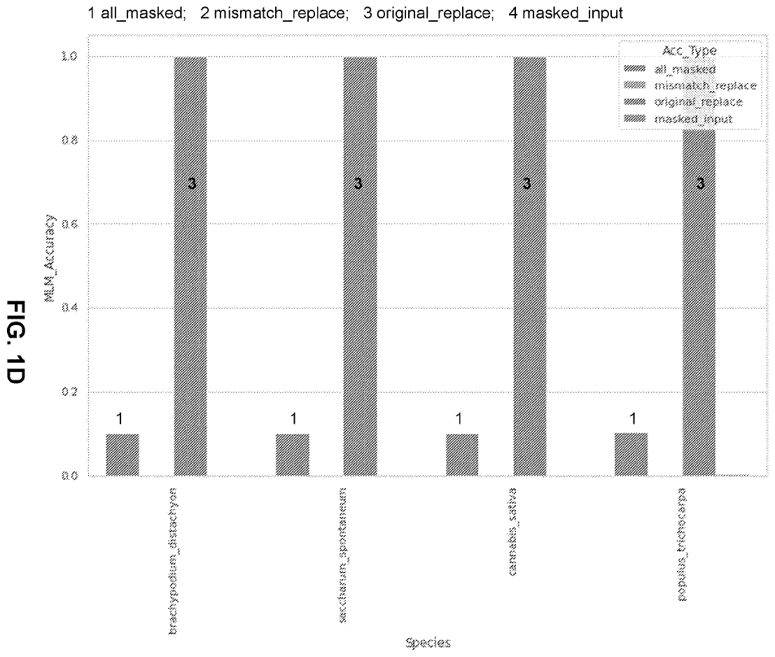

As shown in FIG. 1A, k-mer accuracy for masked tokens in the pretraining species testing set ranged from 0.145 in A. thaliana to 0.53 in Hordeum vulgare. K-mer accuracy varied by task. The accuracy of inferring the presence of an original token was consistently around 1. The accuracy of inferring a masked token ranged from 0.053 in A. thaliana to 0.487 in H. vulgare. The accuracy of identifying and correcting incorrect token replacement ranged from 0.028 in A. thaliana to 0.443 in Hordeum vulgare.

Prediction of masked tokens was also performed using permuted pretraining species testing sequences to maintain the base content properties of each pretraining genome while removing local, contextual sequence signals. For the MLM accuracy metrics, “masked input” refers to the 80% of tokens in which the k-mer was replaced with a random “MASK” token, “mismatch replace” refers to the 10% of tokens in which the k-mer was replaced with a randomly assigned, incorrect (i.e., non-identical) token, and “original replace” refers to the 10% of tokens in which the original token identity was retained.

FIG. 1B provides k-mer accuracy results of permuted sequences in the pretraining species testing dataset. The overall mask accuracy of permutated pretraining sequences was around 0.1. The accuracy of inferring the presence of an original token was consistently around 1, consistent with a trivial strategy of guessing the provided token in the absence of valid contextual information. The accuracy of identifying incorrect token replacement was consistently 0. The accuracy of inferring a masked token ranged from 0.0013 to 0.0047, consistent with the expected frequency based on random guessing of 5-mers under the pretraining species' base contents.

To evaluate generalization of the BIG BIRD deep learning model to entirely new genomes (“pretraining testing species genomes”), polynucleotide sequences from Populus trichocarpa, Brachypodium distachyon, Saccharum spontaneum, and Cannabis sativa (“pretraining testing species sequences”) were encoded as input and masked as described above. FIGS. 1C-1D provide k-mer accuracy results of the pretraining testing species sequences. The overall mask accuracy of the testing species sequences ranged from 0.14 in B. distachyon to 0.21 in Cannabis sativa, which was higher than the permutated testing species sequences. The accuracy of inferring the presence of an original token was consistently around 1. The accuracy of identifying and correcting incorrect tokens ranged from 0.024 in B. distachyon to 0.093 in C. sativa. The accuracy of inferring a masked token ranged from 0.05 in B. distachyon to 0.121 in S. spontaneum.

### Fine-Tuning

Following pretraining of the BIG BIRD backbone, fine-tuning of a fully-connected head layer for polynucleotide expression prediction was performed. For initial fine-tune training, expression data of 6 maize genomes from the Nested Association Mapping (NAM) dataset (doi: 10.1126/science.abg5289) was used, with inputs consisting of putative 2 kb promoters and outputs for 15 maize tissues, including seedling roots and shoots, immature ears, the developing shoot apical meristem, and the developing tassel. All homologs of B73 testing genes were removed from fine-tune training, while homologs of B73 validation genes were retrained for fine-tune validation in each species. Promoter input consisted of polynucleotide sequences 1.85 kb upstream of a putative transcriptional start site (TSS) and 150 bp downstream of the TSS. Input to the expression-predicting head layer consisted of mean-pooled outputs from the final transformer-based backbone layer. The head and transformer backbone layers were permitted to update their weight during this process. Following training on the 6 NAM genomes, final fine-tune training was performed on a set of 41 B73 maize tissues retrieved from the maizeGDB qTeller dataset (doi: 10.1093/bioinformatics/btab604). The training configuration was maintained from the NAM expression prediction task, with the additional constraint that embedding layers and all transformer layers above the final layer were frozen during this fine-tuning stage.

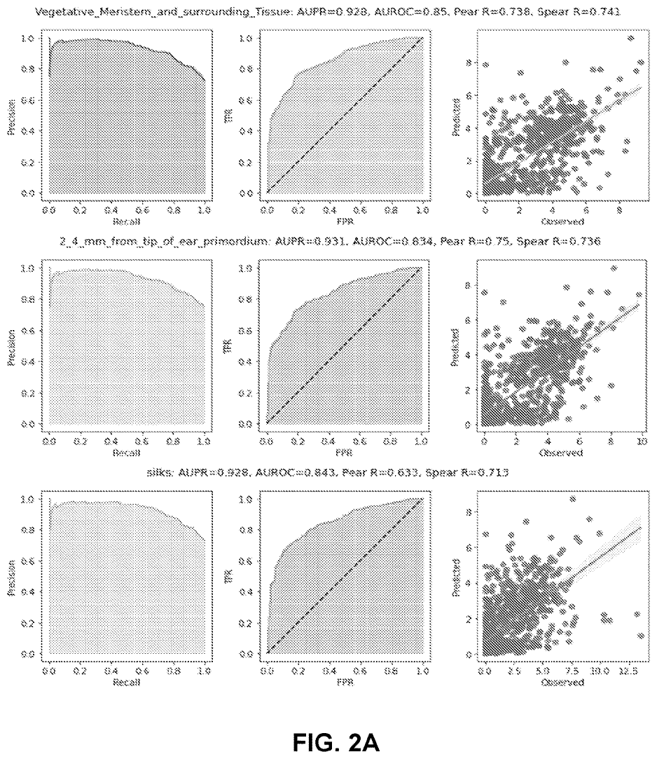

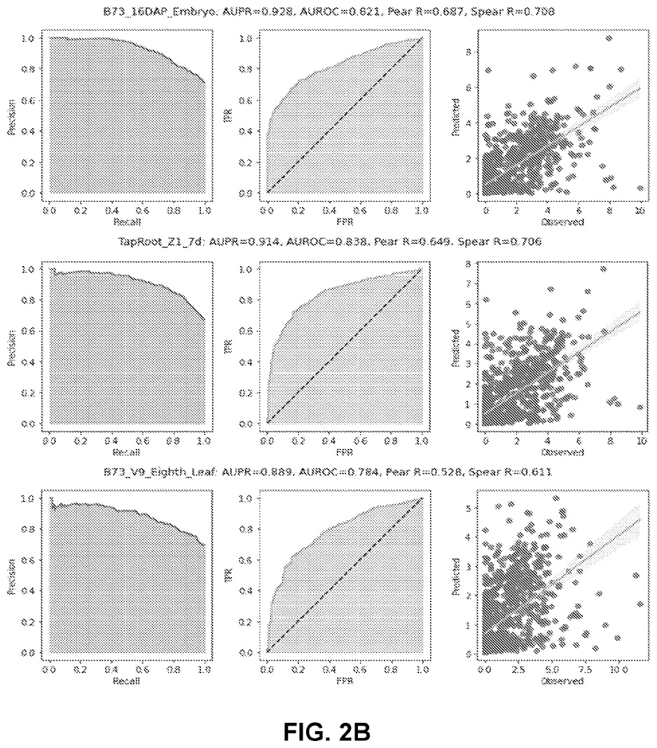

Using the fine-tune testing set of genes, predictive performance in the 41 B73 maize tissues used for fine-tune training was evaluated (FIGS. 2A and 2B). Accuracy, provided as a Pearson correlation between the predicted and observed log 2 (FPKM+1) ranged from 0.53 in the eighth leaf of V9 stage to 0.75 in the 2-4 mm tip of the ear primordium. In FIGS. 2A and 2B, the subplots illustrate testing accuracy metrics for a representative set of 6 tissues used for prediction, including a precision-recall (“PR”) curve (left), a receiver-operator characteristic (“ROC”; middle), and the predicted vs. observed expression on a continuous scale (right). For PR and ROC curves, the observed binary values indicated whether non-zero expression was detected in the respective maize tissue. AUPR=Area Under Precision Recall Curve; AUROC=Area Under Receiver Operator Characteristic; Pear R=Pearson R Correlation; Spear R=Spearman Rank Correlation.

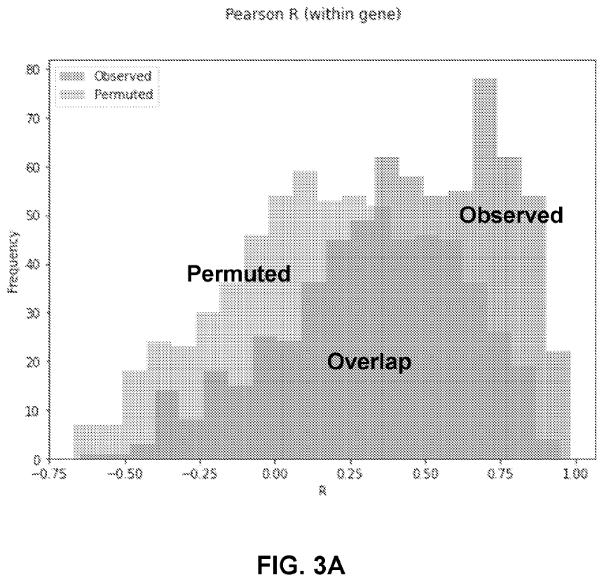

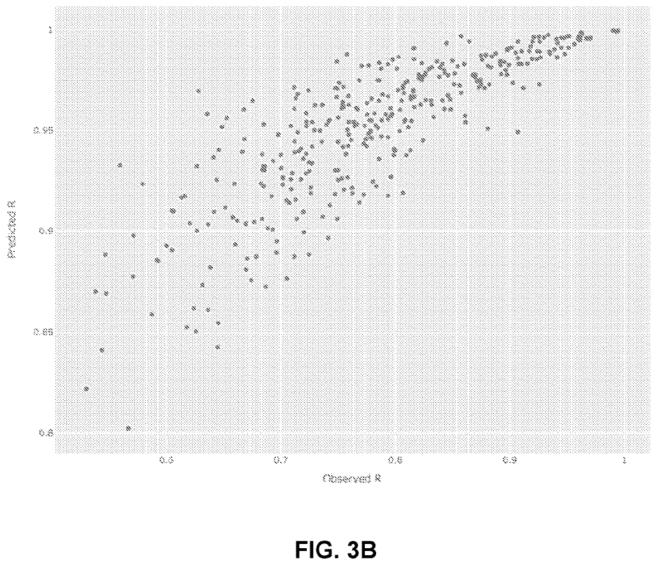

FIG. 3A illustrates distribution of within-gene Pearson R correlations among genes in the fine-tune testing set as observed or after permuting expressed genes among the predicted genes. The permuted distribution therefore indicates the extent to which tissue-biased patterns could be predicted based only on systematic differences among tissue expression datasets.

As shown in FIG. 3A, predicted expression values accurately captured variation in tissue-specific expression with a mean within-gene/among-tissue Pearson correlation of 0.43 across testing set genes. This value is higher than would be expected due to systematic differences between observed tissue expression levels (Mann-Whitney U, p<1e-16), as indicated by the lower correlation of 0.19 between predicted and observed (when the predicted and observed gene sets are permuted relative to one another). FIG. 3B illustrates the relationship between tissue-tissue expression correlations in the predicted fine-tune testing set vs. the expression correlations in the observed fine-tune testing set. As shown in FIG. 3B, predicted vs. observed tissue-tissue correlations associated positively with one another, though the predicted tissue-tissue correlations were generally higher than observed.

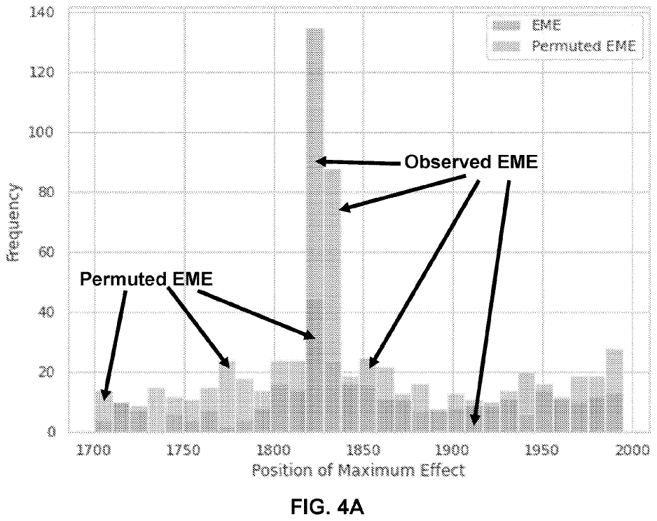

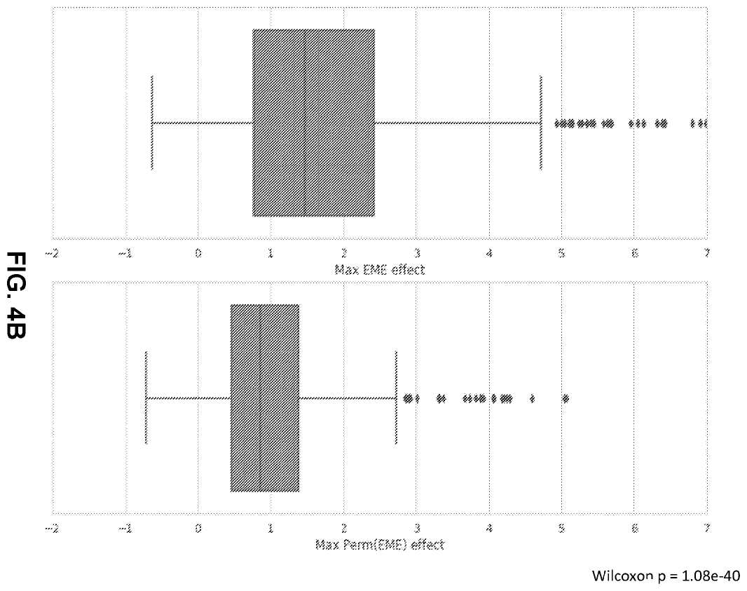

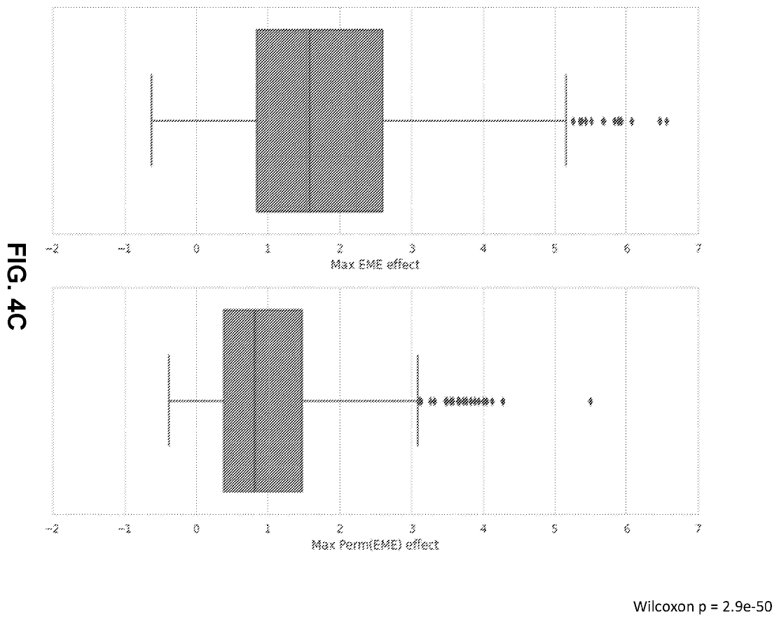

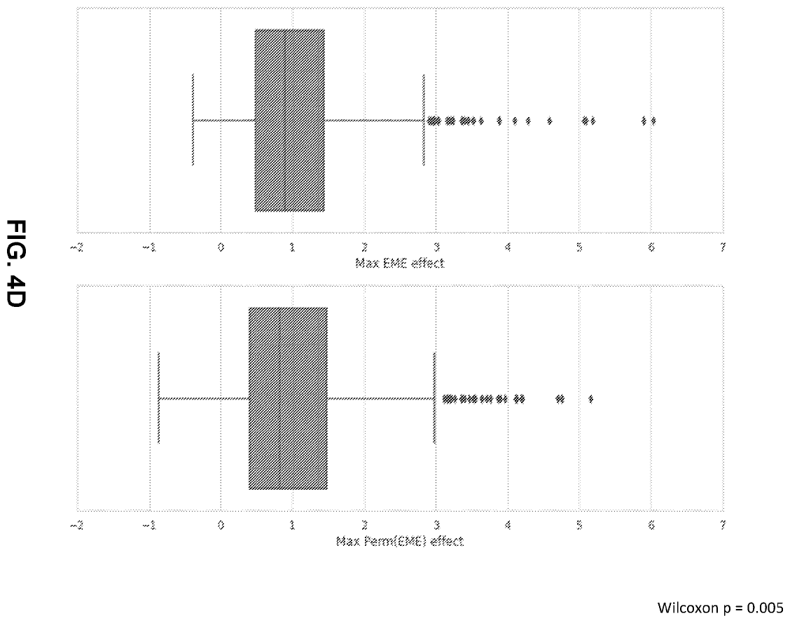

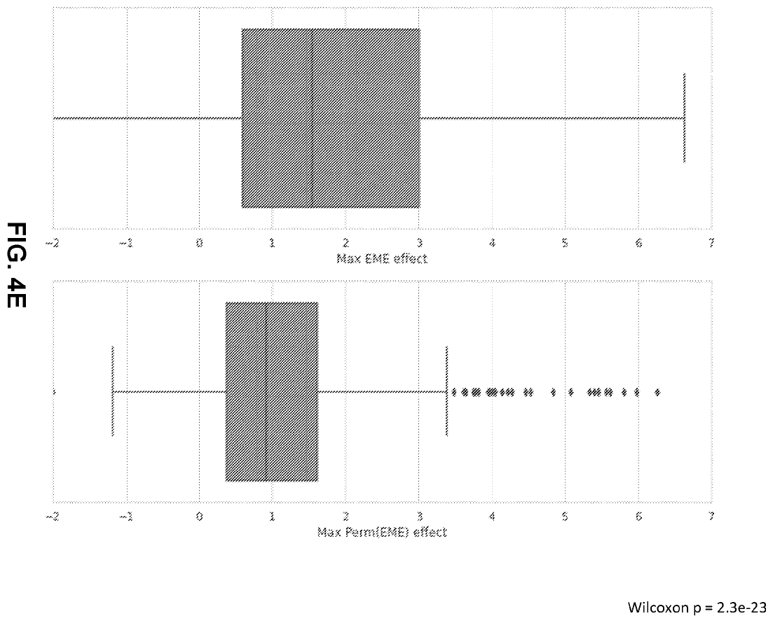

To assess the ability of the final trained expression predictor to infer the impact of sequence modifications on polynucleotide expression, the change in predicted expression was assessed following the insertion of known Expression Modulating Elements (EMEs) into the promoters of the fine-tune testing set. For each EME, the maximum change in predicted expression was calculated across tissues and compared against the maximum change in predicted expression from a permuted version of the EME sequence to control for the effects of both base content and insertion size. Several EMEs were tested. The insertion of a canonical TATA box (i.e., TATATAAA) resulted in a median maximal predicted expression increase of approximately 2.8-fold, with the position of maximal expression increase centered around-30 bp from the putative TSS (FIGS. 4A-4B). In contrast, the median permuted TATA box sequence resulted in a maximal increase of less than 2-fold, which was significantly less than the canonical EME (Wilcoxon p<1.e-16). Additionally, optimal positioning of the permuted EME insertions resulted in low concentration around any single position. Insertions of 2×HSF, 2×TCP, and 1×CMV35S elements also resulted in significant increases in expression relative to their sequence permutations (FIGS. 4C-4F).

FIG. 4A demonstrates the position of maximal effect following the insertion of a canonical TATA box or a permuted TATA box sequence. The putative TSS was positioned at 1850 bp. FIG. 4B illustrates distribution of the maximal changes in predicted expression of a testing set gene following insertion of the canonical TATA box or the permuted TATA box. FIG. 4C illustrates distribution of the maximal changes in predicted expression of a testing set gene following insertion of a dual copy of the TCP element or a dual copy of the permuted TCP sequence. FIG. 4D illustrates distribution of the maximal changes in predicted expression of a testing set gene following insertion of a dual copy of the HSF element or a dual copy of the permuted HSF sequence. FIG. 4E illustrates distribution of the maximal changes in predicted expression of a testing set gene following insertion of a CMV35S 90 bp sequence or a permuted CMV35S 90 bp sequence.

### Example 2: Use of a Deep Neural Network and Genetic Algorithm for Designing Genetic Variants

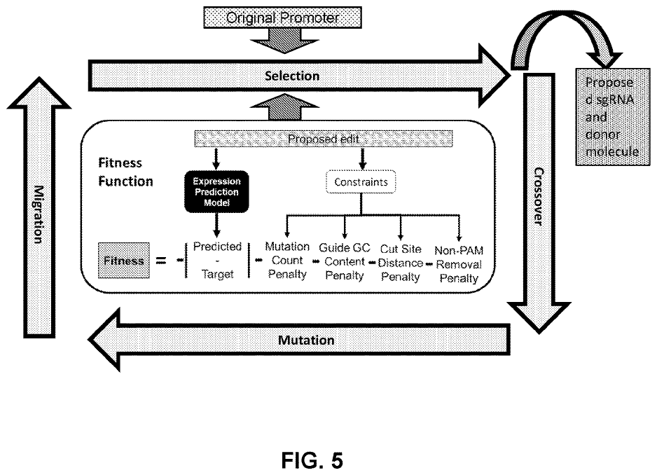

The trained expression predictor from Example 1 was used as part of a genetic algorithm for expression optimization of target genes, given a set of design constraints imposed by a target genome editing system. FIG. 5 is a schematic of the algorithmic design process for a promoter with a modified expression profile. An original- or “reference” promoter is transformed into one or more populations of variant sequences. These populations undergo an in silico evolutionary process comprised of multiple rounds of crossover (recombination between pairs of variant sequences), mutation of variant sequences, migration of variant sequences between populations, and selection of sequences in each population based on a fitness function. The fitness function incentivizes predicted expression profiles closer to a user-specified target, while imposing constraints on the total mutation count, the guide GC content, the distance of mutations from a cut site, and whether the PAM sequence for the selected guide was removed.

Expression optimization was performed to drive promoter expression to target levels, including increased and decreased expression levels relative to wild-type promoter expression. The site-specific genome edits created substitutions within the promoter sequences of target genes by inducing a double-strand break followed by homologous recombination with a donor molecule having the desired substitutions. All edits were constrained to occur upstream of a putative transcription start site (TSS).

Using the genetic algorithm, guide RNA and donor template nucleotide sequences were designed for two different Cas endonucleases, Cas9 endonuclease and Cas12f endonuclease. The primary term in the objective function was the weighted mean of the absolute differences between wild-type and predicted expression for each tissue in the genome modified or edited plants. Weights enabled the attribution of differential importance to the varying tissue types, with full weight on the 2-4 mm and 6-8 mm maize ear primordia. The expression predictor described in Example 1 (i.e., the deep neural network) was used to define part of the genetic algorithm objective function.

Constraints to the quantity, content, and placement of genome edits were imposed through a series of penalties in the genetic algorithm's objective function. To promote parsimony, the number of nucleobase substitutions was penalized with a weight of 0.05. Guide RNA sequences having a GC content below 0.35 or above 0.65 were penalized with a weight of 0.125. Given the known cut sites of the Cas endonucleases used for optimization, a penalty was incurred based on the furthest distance of any substitution position-denoted here as the maximum mutation distance—from the cut site of the Cas endonuclease polypeptide. The maximum mutation distance was calculated based on the closest appropriate protospacer adjacent motif (PAM), Maximum mutation distance was set to 0 if less than or equal to 12, while each unit above 12 added an additional 0.0125 to the penalty term. To avoid re-cutting following homologous recombination with the donor template nucleotide sequence, an additional 0.25 was added to the penalty term if the PAM of the designed guide RNA was not eliminated by the set of substitutions. All proposed substitutions were constrained to fall within a window of 60 bp, though the positioning of this 60 bp window was permitted to vary within the promoter region.

The stages or steps of the genetic algorithm consisted of selection, crossover, mutation, and migration. For selection, the tournament selection process with a tournament size of 10 was used. For each pair of variant sequences selected, two-point crossover was allowed to occur uniformly at random across the nucleotide sequence with a probability of 0.5. During the mutation stage, mutation could occur in two ways. First, with a probability of 0.25, nucleobases were permitted to mutate at random with a probability of 0.025 per base. Second, with a probability of 0.1, the mutation window was permitted to move up to 25 bp in either direction, uniformly at random. The evolving meta-population of potential designs consisted of 5 individual populations, each containing 128 sequences. The migration step then allowed each pair of populations to exchange variant sequences with a probability of 0.01 per sequence, using binomial sampling. Each run of the genetic algorithm was carried forward through 100 generations of in silico evolution. For each promoter design, the sequence with the highest fitness was chosen as a candidate edit. Ultimately, the highest fitness edit meeting all guide constraints was chosen for actual editing in planta.

A total of six designs were constructed, three for use with a Cas9 polypeptide and three for use with a Cas12f polypeptide. All designs were able to modify the predicted expression of the promoter to approximately the level of intended or target expression within the set of constraints provided. For example, substitution of five positions within a 7 bp window of ZMM28 modified the predicted ear primordium expression from log 2 (FPKM+1)=4.9 to a value of 6.3, with a Cas12f donor design inducing modification of the CTTG PAM to a sequence of GTTT. A Cas9 design for BIG GRAIN1 upregulation used 11 substitutions within a window size of 30 to change the predicted expression of log 2 (FPKM+1)=3.0 for the wild-type promoter sequence to a value of 6.05, wherein the donor was designed to modify the PAM from its original CGG sequence to a non-active ATG sequence.

### Example 3: Use of a Deep Neural Network and Genetic Algorithm for Targeted Editing of Distal/Alternative Gene Expression Control Elements

The trained expression predictor from Example 1 can be used as part of a genetic algorithm for expression optimization of target genes, where the design elements and training data can include multiple distal/alternative expression control elements such as for example, distal enhancers, distal silencers, insulator elements, 3′-UTR-miRNA or siRNA binding sites (post-transcriptional regulation), 5′-UTR (uORFs, translational regulation). These alternative/distal editing targets, while subject to some of the design constraints of the employed genome editing system (e.g., Cas9, Cpf1, Cas12f1 and others), also provide additional target regions to modulate expression levels and patterns that are otherwise not exploited in a traditional promoter-region genome editing system. Further, combinations of proximal edits (i.e., within about 1-5 kb of a TATA box of the promoter region) with distal edits can provide additional multiplex genome editing for germplasm improvement.

As described in Example 2, given a set of design constraints imposed by a target genome editing system, having additional targets (e.g., distal targets) to edit, increase the power of whole-genome manipulation for increasing allelic diversity. FIG. 5 is a schematic of the algorithmic design process for a promoter with a modified expression profile. This schematic is readily adapted for providing alternative targets such as for example, distal enhancers, distal silencers, insulator elements, 3′-UTR-miRNA or siRNA binding sites (post-transcriptional regulation), 5′-UTR (uORFs, translational regulation). An original- or “reference” distal regulatory sequence is transformed into one or more populations of variant sequences. These populations undergo an in silico evolutionary process comprised of multiple rounds of crossover (recombination between pairs of variant sequences), mutation of variant sequences, migration of variant sequences between populations, and selection of sequences in each population based on a fitness function. The fitness function incentivizes predicted expression profiles closer to a user-specified target, while imposing constraints on the total mutation count, the guide GC content, the distance of mutations from a cut site, and whether the PAM sequence for the selected guide was removed.

Expression optimization is performed as described in Example 2 to drive expression to target levels, including increased and decreased expression levels relative to wild-type expression. The site-specific genome edits create substitutions within the distal regulatory sequences of target genes by inducing a double-strand break followed by homologous recombination with a donor molecule having the desired substitutions.

### Example 4: Use of a Deep Neural Network and Genetic Algorithm for Targeted Editing Of Genetic Elements Involved in Epigenetic Regulation of Gene Expression

The trained expression predictor from Example 1 can be used as part of a genetic algorithm for expression optimization of target genes, where the design elements and training data can include multiple distal/alternative expression control elements such as for example, distal sequences for lncRNA regulation, epigenetic targeting-methyltransferases, chromatin remodelers, and histone acetyltransferase/methyltransferase. These alternative/distal editing targets, while subject to some of the design constraints of the employed genome editing system (e.g., Cas9, Cpf1, Cas12f1 and others), also provide additional target regions to modulate expression levels and patterns that are otherwise not exploited in a traditional promoter-region genome editing system. Further, combinations of proximal edits (i.e., within about 1-5 kb of a TATA box of the promoter region) with these distal edits can provide additional multiplex genome editing for germplasm improvement.

As described in Example 2, given a set of design constraints imposed by a target genome editing system, having additional targets (e.g., distal targets) to edit, increase the power of whole-genome manipulation for increasing allelic diversity. FIG. 5 is a schematic of the algorithmic design process for a promoter with a modified expression profile. This schematic is readily adapted for providing alternative targets such as for example, distal sequences for lncRNA regulation, epigenetic targeting-methyltransferases, chromatin remodelers, and histone acetyltransferase/methyltransferase. lncRNAs are a class of epigenetic RNA regulators that are involved in epigenetic regulation for example, in the nucleus. The lncRNAs regulate gene transcription by modulating histone or DNA modification by e.g., methylation and acetylation. An original- or “reference” distal regulatory sequence is transformed into one or more populations of variant sequences. These populations undergo an in silico evolutionary process comprised of multiple rounds of crossover (recombination between pairs of variant sequences), mutation of variant sequences, migration of variant sequences between populations, and selection of sequences in each population based on a fitness function. The fitness function incentivizes predicted expression profiles closer to a user-specified target, while imposing constraints on the total mutation count, the guide GC content, the distance of mutations from a cut site, and whether the PAM sequence for the selected guide was removed.

Expression optimization is performed as described in Example 2 to drive expression to target levels, including increased and decreased expression levels relative to wild-type expression. The site-specific genome edits create substitutions within the distal regulatory sequences of target genes by inducing a double-strand break followed by homologous recombination with a donor molecule having the desired substitutions.

### Example 5: Use of a Deep Neural Network and Genetic Algorithm to Identify Motifs Conferring Constitutive Expression of ZmFAD2

This example compares motif identification for constitutive expression of a target gene using the trained expression predictor of Example 1 with a comparative genomics method. In the comparative genomics method, to identify motifs underlying constitutive expression of ZmFAD2 (Zm00001d017840), orthologs were selected from Phytozome (v13) based on previously defined criteria. The promoters, 5′ UTRs, and first introns of five orthologous Fad2 genes (including ZmFAD2) were selected for comparative and MEME analysis (Table 1). First, selected sequences were subjected to MEME analysis tool from ‘The MEME Suite’ (Bailey et al. 2015) to identify orthologous blocks with an upper limit of 50 nucleotides. These orthologous blocks were annotated on the sequences and used to aid alignment of the divergent promoter/intron regions. Once aligned, conserved motifs known to confer constitutive expression were scanned and annotated using a proprietary motif database with a 91% cutoff value. Next, promoter/intron sequences were subjected to an additional MEME analysis with an expected motif size of 6-15 nucleotide length with parameters ‘anr’ (any number of repetitions) and up to 25 predicted motifs. The data from these three inputs were correlated to identify putative critical motifs for constitutive expression in ZmFAD2. These results were compared against motifs predicted by the trained expression predictor of Example 1. The expression predictor was used to predict expression resulting from sequential 10 bp deletions within the promoter and adjacent 5′UTR sequence, and the region with the highest predicted negative impact on expression was selected for further study. Two motifs were predicted by both approaches to have high probability as critical functionality for constitutive expression, ‘AGCAA’ in the predicted 5′ UTR and ‘CCGCTTTTAAAT’, the latter of which contains a core ‘Dof’ transcription factor motif and ‘TATA’-like sequence.

TABLE 1
Five orthologous FAD2 promoter/intron regions selected
for comparative genomics and motif analysis.

| Gene Name | Species | Phytozome Genome | Gene ID |
|---|---|---|---|
| ZmFAD2 | Zea mays | Z. mays RefGen_V4 | Zm00001d017840 |
| SbFAD2 | Sorghum bicolor | S. bicolor v3.1.1 | Sobic.004G260800 |
| SiFAD2 | Setaria italica | S. italica v2.2 | Seita.1G296200 |
| PhFAD2 | Panicum hallii | P. hallii v3.2 | Pahal.1G371800 |
| PvFAD2 | Paspalum vaginatum | P. vaginatum v3.1 | Pavag04G254800 |

1. An artificial intelligence model-mediated method for editing a plant genome, the method comprising:
(a) providing an artificial intelligence (AI) model with a first dataset, the first dataset comprising a reference nucleotide sequence of at least one plant regulatory element;
(b) providing the AI with a second dataset, the second dataset comprising a plurality of variant nucleotide sequences of the reference nucleotide sequence;
(c) predicting one or more expression profiles for each variant nucleotide sequence of the plurality of variant nucleotide sequences relative to expression of the reference nucleotide sequence;
(d) calculating a first fitness score for each variant nucleotide sequence, wherein the first fitness score reflects the degree to which a predicted expression profile for a variant nucleotide sequence meets a target expression profile, and wherein the first fitness score incorporates one or more constraints that alter the suitability of the variant nucleotide sequence based on a genome editing system;
(e) selecting at least one final variant nucleotide sequence from the plurality of variant nucleotide sequences; and
(f) editing the plant genome.

2. The method of claim 1, wherein prior to selecting the at least one final variant nucleotide sequence, the method further comprises:
selecting a subset of variant nucleotide sequences based on the first fitness score for each variant nucleotide sequence of the plurality of variant nucleotide sequences;
providing the AI model with a third dataset, the third dataset comprising the subset of variant nucleotide sequences, wherein each variant nucleotide sequence of the subset of variant nucleotide sequences comprises one or more additional mutations not found in the plurality of variant nucleotide sequences of the second dataset or results from a recombination of two or more of the variant nucleotide sequences from the second dataset; and
optionally repeating the following until a variant nucleotide sequence of the subset of variant nucleotide sequences that meets a target fitness score is identified:
predicting one or more expression profiles for each variant nucleotide sequence of the plurality of variant nucleotide sequences relative to expression of the reference nucleotide sequence;
calculating a first fitness score for each variant nucleotide sequence;
selecting a subset of variant nucleotide sequences based on the first fitness score for each variant nucleotide sequence of the plurality of variant nucleotide sequences; and
providing the AI model with a third dataset, the third dataset comprising the subset of variant nucleotide sequences, wherein each variant nucleotide sequence of the subset of variant nucleotide sequences comprises one or more additional mutations not found in the plurality of variant nucleotide sequences of the second dataset or results from a recombination of two or more of the variant nucleotide sequences from the second dataset,
wherein the variant nucleotide sequence that meets the target fitness score is selected as a final variant nucleotide sequence.

3. The method of claim 1, wherein the reference nucleotide sequence is a native or a wild-type nucleotide sequence of the at least one plant regulatory element.

4. The method of claim 1, wherein editing the plant genome comprises editing a target regulatory element nucleotide sequence in a plant cell such that the target regulatory element nucleotide sequence aligns with the final variant nucleotide sequence.

5. The method of claim 1, wherein the genome editing system comprises a Cas endonuclease and a guide polynucleotide and editing the target regulatory element nucleotide sequence comprises providing the plant cell with the Cas endonuclease and the guide polynucleotide to introduce at least one site-specific modification in the target regulatory element nucleotide sequence resulting in the variant nucleotide sequence.

6. The method of claim 5, wherein the genome editing system further comprises a donor DNA.

7. The method of claim 5, wherein editing the target regulatory element nucleotide sequence comprises introducing at least one nucleotide insertion, at least one nucleotide deletion, at least one nucleotide substitution, or a combination thereof to achieve the final variant nucleotide sequence.

8. The method of claim 5, wherein the Cas endonuclease is a Cas12 endonuclease or a Cas9 endonuclease.

9. The method of claim 1, wherein the genome editing system comprises a base editing agent and a plurality of guide polynucleotides and editing the target regulatory element nucleotide sequence comprises providing the plant cell with the base editing agent and the plurality of guide polynucleotides to introduce a plurality of nucleobase edits in the target regulatory element nucleotide sequence resulting in the variant nucleotide sequence.

10. The method of claim 9, wherein the base editing agent comprises a deactivated Cas endonuclease (dCas) complexed to a deaminase.

11. The method of claim 10, wherein the deactivated Cas endonuclease is a dCas12f endonuclease or a dCas9 endonuclease and the deaminase is a cytosine deaminase or an adenosine deaminase.

12. The method of claim 1, wherein the one or more constraints impose a penalty value on the fitness score.

13. The method of claim 11, wherein the one or more constraints are selected from functions penalizing mutation count, a parsimony constraint, failure to remove a protospacer adjacent motif (PAM) site, lack of a properly positioned PAM site, suboptimal GC content of one or more guide polynucleotides, and distance of a site-specific modification from a DNA break in the target regulatory element nucleotide sequence.

14. The method of claim 1, wherein the AI model is a natural language processing model, a transformer-based neural network, a convolutional neural network, or a combination thereof.

15. An artificial intelligence method for predicting expression modifications due to genetic variants, the method comprising:
(a) providing an artificial intelligence (AI) model with a first dataset, the first dataset comprising a reference nucleotide sequence of at least one plant regulatory element;
(b) providing the AI model with a second dataset, the second dataset comprising a plurality of variant nucleotide sequences of the reference nucleotide sequence;
(c) predicting one or more expression profiles of each variant nucleotide sequence of the plurality of variant nucleotide sequences relative to expression of the reference nucleotide sequence; and
(d) calculating a first fitness score for each variant nucleotide sequence, wherein the first fitness score reflects the degree to which a predicted expression profile for a variant nucleotide sequence meets a target expression profile, and wherein the first fitness score incorporates one or more constraints that alter the suitability of the variant nucleotide sequence.

16. The method of claim 15, further comprising:
selecting a subset of variant nucleotide sequences based on the first fitness score for each variant nucleotide sequence of the plurality of variant nucleotide sequences;
providing the AI model with a third dataset, the third dataset comprising the subset of variant nucleotide sequences, wherein each variant nucleotide sequence of the subset of variant nucleotide sequences comprises one or more additional mutations not found in the plurality of variant nucleotide sequences of the second dataset or results from a recombination of two or more of the variant nucleotide sequences from the second dataset; and
optionally repeating the following until a variant nucleotide sequence of the subset of variant nucleotide sequences that meets a target fitness score is identified:
predicting one or more expression profiles for each variant nucleotide sequence of the plurality of variant nucleotide sequences relative to expression of the reference nucleotide sequence;
calculating a first fitness score for each variant nucleotide sequence;
selecting a subset of variant nucleotide sequences based on the first fitness score for each variant nucleotide sequence of the plurality of variant nucleotide sequences; and
providing the AI model with a third dataset, the third dataset comprising the subset of variant nucleotide sequences, wherein each variant nucleotide sequence of the subset of variant nucleotide sequences comprises one or more additional mutations not found in the plurality of variant nucleotide sequences of the second dataset or results from a recombination of two or more of the variant nucleotide sequences from the second dataset,
wherein the variant nucleotide sequence that meets the target fitness score is selected as a final variant nucleotide sequence.

17. The method of claim 15, wherein the reference nucleotide sequence is a native or a wild-type nucleotide sequence of the plant regulatory element.

18. The method of claim 15, wherein the one or more constraints impose a penalty value on the fitness score.

19. The method of claim 18, further comprising defining the one or more constraints based on a genome editing system.

20. The method of claim 19, wherein the genome editing system comprises:
(i) a Cas endonuclease and a guide polynucleotide; or
(ii) a base editing agent and a plurality of guide polynucleotides.

21. The method of claim 20, wherein the base editing agent comprises a deactivated Cas endonuclease (dCas) complexed to a deaminase.

22. The method of claim 20, wherein the Cas endonuclease is a Cas12f endonuclease or a Cas9 endonuclease.

23. The method of claim 21, wherein the dCas endonuclease is a dCas12f endonuclease or a dCas9 endonuclease and the deaminase is a cytosine deaminase or an adenosine deaminase.

24. The method of claim 20, wherein the one or more constraints are selected from the group functions penalizing mutation count, a parsimony constraint, failure to remove a protospacer adjacent motif (PAM) site, lack of a properly positioned PAM site, suboptimal GC content of one or more guide polynucleotides, and distance of a site-specific modification from a DNA break in the target regulatory element nucleotide sequence.

25. The method of claim 15, wherein the AI model is a natural language processing model, a transformer-based neural network, a convolutional neural network, or a combination thereof.

26. An artificial intelligence model-mediated method for breeding genetically modified plants, the method comprising:
calculating a fitness score for each of a plurality of variant nucleotide sequences of a plant regulatory element, wherein calculating the fitness score comprises providing an artificial intelligence (AI) model with a first dataset comprising a reference nucleotide sequence of the plant regulatory element and a second dataset comprising the plurality of variant nucleotide sequences of the plant regulatory element and predicting one or more expression profiles of each of the plurality of variant nucleotide sequences relative to expression of the reference nucleotide sequence;
selecting a variant nucleotide sequence from the plurality of variant nucleotide sequences based on the fitness score;
providing a plant cell with a genome editing system that edits a target regulatory element nucleotide sequence of the plant cell such that the target regulatory element nucleotide sequence aligns with the selected variant nucleotide sequence;
regenerating a genetically modified first plant from the plant cell; and
crossing the genetically modified first plant with a second plant to produce a population of genetically modified plants.

27. The method of claim 26, wherein prior to selecting the variant nucleotide sequence from the plurality of variant nucleotide sequences, the method further comprises:
(a) predicting one or more expression profiles for each variant nucleotide sequence of the plurality of variant nucleotide sequences relative to expression of the reference nucleotide sequence;
(b) calculating an initial fitness score for each of the plurality of variant nucleotide sequences, wherein the fitness score reflects the degree to which a predicted expression profile for a variant nucleotide sequence meets a target expression profile, and wherein the fitness score incorporates one or more constraints that alter the suitability of the variant nucleotide sequence based on a genome editing system;
(c) selecting a subset of variant nucleotide sequences based on the initial fitness score for each variant nucleotide sequence of the plurality of variant nucleotide sequences;
(d) providing the AI model with a third dataset, the third dataset comprising the subset of variant nucleotide sequences, wherein each variant nucleotide sequence of the subset of variant nucleotide sequences comprises one or more additional mutations not found in the plurality of variant nucleotide sequences in the second dataset or results from a recombination of two or more of the variant nucleotide sequences from the second dataset;
(e) optionally repeating (a)-(d) until a variant nucleotide sequence of the subset of variant nucleotide sequences that meets a target fitness score is identified; and
(f) selecting at least one final variant nucleotide sequence from the subset of variant nucleotide sequences, wherein the at least one final variant nucleotide sequence meets the target fitness score.

28. The method of claim 26, wherein the reference nucleotide sequence is a native or a wild-type nucleotide sequence of the plant regulatory element.

29. The method of claim 26, wherein the genome editing system comprises a Cas endonuclease and a guide polynucleotide that introduce at least one site-specific modification in the target regulatory element nucleotide sequence of the plant cell resulting in the selected variant nucleotide sequence.

30. The method of claim 29, wherein the genome editing system further comprises a donor DNA.

31. The method of claim 29, wherein the at least one site-specific modification comprises an insertion, a deletion, a substitution, or a combination thereof.

32. The method of claim 29, wherein the Cas endonuclease is a Cas12f endonuclease or a Cas9 endonuclease.

33. The method of claim 26, wherein the genome editing system comprises a base editing agent and a plurality of guide polynucleotides that introduce a plurality of nucleobase edits in the target regulatory element nucleotide sequence.

34. The method of claim 33, wherein the base editing agent comprises a deactivated Cas endonuclease (dCas) complexed to a deaminase.

35. The method of claim 34, wherein the deactivated Cas endonuclease is a dCas12f endonuclease or a dCas9 endonuclease and the deaminase is a cytosine deaminase or an adenosine deaminase.

36. The method of claim 29, wherein calculating the fitness score further comprises imposing a penalty value on the fitness score based on one or more constraints.

37. The method of claim 36, wherein the one or more constraints are selected from the group functions penalizing mutation count, a parsimony constraint, failure to remove a protospacer adjacent motif (PAM) site, lack of a properly positioned PAM site, suboptimal GC content of one or more guide polynucleotides, and distance of a site-specific modification from a DNA break in the target regulatory element nucleotide sequence.

38. The method of claim 26, wherein the AI model is a natural language processing model, a transformer-based neural network, a convolutional neural network, or a combination thereof.

39.-45. (canceled)

46. A system for predicting expression of genetic variants, the system comprising a computer-readable medium comprising an artificial intelligence (AI) model, wherein the AI is configured to:
calculate a fitness score for each of a plurality of variant nucleotide sequences of a plant regulatory element, wherein calculating the fitness score comprises providing the AI model with a first dataset comprising a reference nucleotide sequence of the plant regulatory element and a second dataset comprising the plurality of variant nucleotide sequences of the plant regulatory element and predicting one or more expression profiles of each of the plurality of variant nucleotide sequences relative to expression of the reference nucleotide sequence; and
select a variant nucleotide sequence from the plurality of variant nucleotide sequences based on the fitness score.

47. The system of claim 46, wherein prior to selecting the variant nucleotide sequence from the plurality of variant nucleotide sequences, the system is configured to:
(a) predict one or more expression profiles for each variant nucleotide sequence of the plurality of variant nucleotide sequences relative to expression of the reference nucleotide sequence;
(b) calculate an initial fitness score for each of the plurality of variant nucleotide sequences, wherein the fitness score reflects the degree to which a predicted expression profile for a variant nucleotide sequence meets a target expression profile, and wherein the fitness score incorporates one or more constraints that alter the suitability of the variant nucleotide sequence based on a genome editing system;
(c) select a subset of variant nucleotide sequences based on the initial fitness score for each variant nucleotide sequence of the plurality of variant nucleotide sequences;
(d) provide the AI model with a third dataset, the third dataset comprising the subset of variant nucleotide sequences, wherein each variant nucleotide sequence of the subset of variant nucleotide sequences comprises one or more additional mutations not found in the plurality of variant nucleotide sequences in the second dataset or results from a recombination of two or more of the variant nucleotide sequences from the second dataset;
(e) optionally repeat (a)-(d) until a variant nucleotide sequence of the subset of variant nucleotide sequences that meets a target fitness score is identified; and
(f) select at least one final variant nucleotide sequence from the subset of variant nucleotide sequences, wherein the at least one final variant nucleotide sequence meets the target fitness score.

48. The system of claim 46, further comprising a computing device comprising a processor.

49. The system of claim 46, wherein the reference nucleotide sequence is a native or a wild-type nucleotide sequence of the plant regulatory element.

50. The system of claim 46, wherein the AI model incorporates one or more constraints to calculate the fitness score.

51. The system of claim 50, wherein the one or more constraints are based on a genome editing system and impose a penalty value on the fitness score.

52. The system of claim 51, wherein the genome editing system comprises a Cas endonuclease, a guide polynucleotide, and optionally a donor DNA.

53. The system of claim 52, wherein the Cas endonuclease is a Cas12f endonuclease or a Cas9 endonuclease.

54. The system of claim 51, wherein the genome editing system comprises a base editing agent and a plurality of guide polynucleotides.

55. The system of claim 54, wherein the base editing agent comprises a deactivated Cas endonuclease (dCas) complexed to a deaminase.

56. The system of claim 55, wherein the deactivated Cas endonuclease is a dCas12f endonuclease or a dCas9 endonuclease and the deaminase is a cytosine deaminase or an adenosine deaminase.

57. The system of claim 54, wherein the selected variant nucleotide sequence comprises nucleobase edits for multiplex base editing of a plant genome.

58. The system of claim 51, wherein the selected variant nucleotide sequence comprises at least 10 nucleobase edits, alternatively at least 100 nucleobase edits, alternatively at least 1000 nucleobase edits.

59. The system of claim 50, wherein the one or more constraints are selected from the group functions penalizing mutation count, a parsimony constraint, failure to remove a protospacer adjacent motif (PAM) site, lack of a properly positioned PAM site, suboptimal GC content of one or more guide polynucleotides, and distance of a site-specific modification from a DNA break in the target regulatory element nucleotide sequence.

60. The system of claim 46, wherein the AI model is a natural language processing model, a transformer-based neural network, a convolutional neural network, or a combination thereof.

61.-100. (canceled)
